(19) United States
(12) Patent Application Publication
(10) Pub. No.: US 2008/0199368 A1
(43) Pub. Date: Aug. 21, 2008

(54) Night soil drying treatment apparatus

(76) Inventors: Masahiro Kanno, Tokyo (JP); Yasuo Kurihara, Tokyo (JP)

Correspondence Address: FLYNN THIEL BOUTELL & TANIS, P.C., 2026 RAMBLING ROAD, KALAMAZOO, MI, 49008-1631, US

(21) Appl. No.: 11/707,772

(22) Filed: Feb. 16, 2007

(51) Int. Cl. A61L 9/00; B08B 3/02; B08B 5/00

(52) U.S. Cl. 422/173; 134/184; 134/198; 15/316.1

(57) ABSTRACT

Foreign objects such as carbide and so forth are prevented from being adhered, solidified, accumulated in the interior of a drying cauldron every time drying treatment of night soil is completed, and heat transfer by a cauldron bottom heater caused by the accumulation of foreign objects is not prevented, thereby smoothly implementing treatment of next cycle and succeeding treatment. In a night soil drying treatment apparatus wherein when night soil inside the night soil tank is sucked up and thrown into a drying cauldron and a load of night soil to be treated is stored in the drying cauldron, the drying cauldron is heated to evaporate night soil in the drying cauldron, and completing night soil drying treatment upon completion of heating and evaporating treatment by a predetermined time, there is provided a washing mechanism for removing adherents onto the interior of the drying cauldron for jetting water toward the interior of the drying cauldron every time one cycle of night soil treatment is completed, thereby automatically washing the interior of the drying cauldron to remove the adherents onto the interior of the drying cauldron.

### FIELD OF THE INVENTION

The invention relates to a treatment apparatus for heating and drying night soil at the outdoors or a transport facilities such as a ship, a train, having no purification treatment apparatus, and places having no waste water treatment facility such as an inside of a tunnel, a riverbed, a construction site, and so forth where a vacuum truck does not enter because it is closed from an outside, particularly, to a drying cauldron inner cleaning mechanism and a catalyst cleaning mechanism of a night soil drying treatment apparatus for automatically cleaning an interior of the drying cauldron and a catalyst mechanism in the treatment apparatus for transferring night soil from a tank which stored therein night soil to a drying cauldron, thereby drying it and deodorizing air by the catalyst mechanism.

### BACKGROUND OF THE INVENTION

Night soil excreted from a human body is discharged into a drain from a flushing toilet and so forth in a general house, or it is temporarily stored in a purification tank to be purified, then discharged into a river. However, in the case where an event is to be held outdoors, for example, in a sports meeting, a trade fair, a site such as a meeting where many people go out, or in a disaster area caused by wind and snow, a flood, an earth quake and so forth, physiological phenomena of participants have been solved by providing a temporary toilet.

As mentioned above, according to treatment of night soil which has been conventionally made outdoors or at the place having no purification facility, a movable temporary toilet has been used. However, most of them has a structure to have a toilet vessel for temporarily storing night soil wherein night soil excreted from a human body has been accumulated therein as it is. Accordingly, night soil stored in the toilet vessel has been collected by a tank truck used for collecting night soil and so forth after the temporary toilet is used, and the collected night soil has to be transferred to a night soil treatment facility. Accordingly, treatment after collecting night soil is needed, which takes a lot of trouble and unhygienic involved in post-treatment.

In transportation facilities such as a train, a bus, a ship and so forth that are moving for a long distance, they have a tank for exclusively used in storage of excreted night soil provided therein, wherein night soil is stored in the tank and subjected to deodorizing treatment by chemicals and so forth, then they have been conventionally collected by the tank truck used for collecting night soil at the terminal station or way station.

According to treatment in the most conventional temporary toilet or movable transportation facilities, night soil is stored in a state as it is excreted from a human body, and collected thereafter. Accordingly, it has to be said that either of a storage method, collection method or treatment method is not modernized and very unhygienic. Accordingly, if the temporary toilet has been used over the long term, the excreted night soil remains in the tank, causing the generation of an offensive odor. Further, since a cleaning operation of the temporary toilet after use thereof is disliked by an operator, it is not preferable in terms of modernization of a maintenance of the temporary toilet or a toilet of the transportation facilities.

Under such circumstances, there have been conventionally conceived various methods for treating night soil hygienically. For example, there is a method for throwing night soil together with chemicals in the temporary toilet, thereby subjecting night soil to sterilizing and deodorizing treatment. This method has been frequently employed by transportation facilities such as a bullet trains and so forth, but it falls into disfavor to a user, because if night soil containing chemicals is circulated between the tank and a toilet stool, water flowing in the toilet stool is soiled during a use over the long term and an offensive odor is generated. Further, since the tank is made empty to be ready for next use, it is necessary to throw large quantity of chemicals in the tank, resulting in the occurrence of a drawback to increase a cost of chemicals.

Further, there is conceived a method for storing night soil in a bag made of vinyl and so forth to be packed, thereby preventing emission of an odor. However, the vinyl bag having an extensive wide area has to be used by one time excretion, leading to the increase of a cost of treatment, and the later requirement of treatment for separating night soil from the vinyl bag. According to this method, treatment until packing is relatively easy but post-treatment is troublesome and a treatment facility becomes large scaled.

Further, there is conceived a method of storing excreted night soil in an airtight evaporation container, and heating night soil directly by a thermal power of a burner, thereby evaporating night soil. This method, for example, is known from JP No. 45-17236B, No. 49-2545B, No. 50-3149B, JP 52-58239A, No. 53-110268A and No. 55-165415A. However, according to the methods disclosed in these publications, since flame of the burner is jetted toward night soil to evaporate night soil from the surface thereof, moisture which is a major component of night soil cannot be efficiently evaporated, thereby requiring much energy for completely treating night soil.

With the structure as disclosed in these publications, cleaning of the evaporation container after evaporating night soil is not implemented so that residues which have not evaporated from night soil are accumulated on the bottom of the evaporation container during a long time use, so that thermal efficiency becomes degraded. In these cases, the apparatus has to be disassembled to implement maintenance thereof every time the residues are cleaned, resulting in the occurrence of a drawback of taking time and cost in the maintenance thereof.

There have been many problems in treatment of night soil in the temporary toilet, as mentioned above, so that excreted night soil is hardly treated completely in the temporary toilet. To meet a social demand, the inventor who is the same as the present applicant of this application has proposed a night soil treatment apparatus capable of evaporating moisture which is a major component of night soil by heating night soil. The proposed night soil treatment apparatus is used by being provided on a temporary toilet, wherein an airtight drying cauldron (heat-resistant evaporation container) for storing night soil is provided in the apparatus, and a rotatable stirring blade is journalled in the drying cauldron, and a plurality of heat accumulators which are rotated together with night soil while the stirring blade is rotated are stored in the drying cauldron.

With the structure, when the drying cauldron is heated from outside to heat night soil in the drying cauldron, and the stirring blade is rotated at the same time when night soil is mixed to thereby rapidly evaporate moisture of a night soil. At the time of evaporation of moisture, spherical heat accumulators are rotated on the bottom of the dying cauldron so that a temperature of night soil is uniformly increased and heat generated thereby is transferred to night soil, thereby increasing heating speed (e.g. disclosed in Japanese Patent Application No. 63-124150).

This mechanism is basic, and the same inventor has sequentially proposed an improvement of the night soil treatment apparatus. In the invention disclosed in Japanese Patent Application No. 2-164594A, there is disclosed a structure in which a pipe for feeding air under pressure into a drying cauldron and a discharge air pipe are connected to each other, and a dust collecting device and a condenser are connected to the discharge air pipe. According to this mechanism, moisture contained in night soil and evaporated inside the drying cauldron is frozen and collected, and it is circulated to be used as flushing water while dust remaining in the drying cauldron is sucked together with air and collected after night soil is dried, and it can be separated from air by a dust collecting device. With the structure of this mechanism, moisture can be collected from night soil thrown in the drying cauldron while dust which remains in the drying cauldron and is not evaporated after night soil is dried can be cleaned, so that the night soil treatment apparatus can be continuously used.

Further, according to the invention disclosed in Japanese Patent Application No. 2-411577, the night soil treatment apparatus is unitized and easily installed on the temporary toilet. This mechanism is structured that the structure of the drying cauldron is formed cylindrically and night soil can be thrown in the drying cauldron from the side surface thereof. To this end, it is not necessary to dispose the toilet stool on the upper portion of the drying cauldron, thereby lowering a height of the apparatus.

According to the invention disclosed in Japanese Patent Application No. 2-412559, a storage vessel for storing urine which is collected by a urine stool is provided, and a jet pipe is connected between the storage vessel and a toilet stool. This is structured such that feces and urine are excreted into a toilet stool while urine is collected by the urine stool, wherein when the feces and urine are thrown in the drying cauldron, urine is jetted from the storage vessel toward the urine stool, and the urine can be thrown in the drying cauldron together with the feces and urine. Accordingly, the toilet stool can be cleaned by urine, so that even a place where water works cannot be installed, the temporary toilet can be converted into a flush toilet.

Further, according to the invention disclosed in Japanese Patent Application No. 3-067538, there is disclosed a mechanism wherein a liquid surface sensor is inserted into the drying cauldron from the side surface thereof, and a position of a liquid surface of a night soil which has been thrown into the drying cauldron can be always detected. According to this mechanism, the height of the liquid surface of night soil is detected so as to decide to prevent the capacity of evaporating treatment of night soil from being lowered, which is caused by temporarily throwing a large quantity of night soil in the drying cauldron, resulting in preventing the generation of trouble caused by overflow of night soil.

According to the invention disclosed in Japanese Patent Application No 3-189280, a re-heating box in which a heater is housed is disposed between a blower and a catalyst box, and a bypass is provided between a pipe connecting a drying cauldron and a dust collecting device and the catalyst box. With this structure, air from the drying cauldron is flown into the catalyst box by the bypass when night soil is subjected to evaporating treatment, thereby enhancing the flow of air efficiently. Further, when the drying cauldron is subjected to cleaning treatment, the bypass is closed so as to cause air containing dust to be flown in the dust collecting device as it is.

Further, the invention disclosed in Japanese Patent Application No. 3-189281 is an improvement of the invention disclosed in Japanese Patent Application No. 3-189280, wherein a pipe of a bypass is connected to a pipe for connecting a drying cauldron and a dust collecting device, and a terminal end of the pipe of the bypass is connected to a negative pressure side of an ejector while a catalyst box is connected to the ejector. With this structure, there is an effect that when the pipe of the bypass is opened, air inside the drying cauldron is forcibly sucked by the ejector to be flown in the catalyst box so that flowing efficiency of air in the drying cauldron is enhanced, thereby promoting evaporation.

According to the invention disclosed in Japanese Patent Application No. 3-265237, a dust collection box is connected to a discharge air pipe of a drying cauldron, and a dust bag formed of paper, cloth and so forth is housed in the dust collection box. With this structure, air containing dust is caused to flow in the dust bag when the drying cauldron is cleaned, whereby air alone is passed through the dust bag while dust is separated from air. There is an effect that dust can be reliably separated compared with a cyclone type dust collecting device.

Further, according to the invention disclosed in Japanese Patent Application No. 5-031170, a toilet stool and a night soil treatment apparatus are separated from each other wherein night soil thrown in the toilet stool is stored in a tank, then it can be subjected to a continuous evaporating drying treatment in a batch system. In the night soil treatment apparatus, there is formed an opening through which night soil is thrown at the upper portion of a substantially spherical drying cauldron which is rotatable by a horizontal shaft, and spherical heat accumulators are housed inside the drying cauldron, and further a burner for heating the bottom of the drying cauldron is formed. With the structure, night soil thrown in the drying cauldron is evaporated and dried by a heat of the burner, and night soil can be stirred by the heat accumulators by swinging the drying cauldron during this evaporating drying treatment while dust can be dropped through the opening by rotating the drying cauldron after night soil is dried.

The invention disclosed in Japanese Patent Application No. 5-121968 is structured that a switching valve is intervened between a blower pipe of a drying cauldron and a discharge air pipe, wherein a dust collection box and a blower can be connected in series to the drying cauldron via the switching valve. With this structure, there is advantage that air is circulated inside the night soil treatment apparatus when the drying cauldron is cleaned so that a dust collecting efficiency is enhanced.

The invention disclosed in Japanese Patent Application No. 5-125360 is structured that a high frequency coil is disposed on the lower surface of a drying cauldron, thereby causing the high frequency coil to generate an electromagnetic wave by supplying a high frequency power. Since the high frequency electromagnetic wave is applied to the drying cauldron, night soil is not heated directly but the drying cauldron per se generates heat to heat night soil, so that there is an effect of reducing thermal loss.

According to the invention disclosed in Japanese Patent Application No. 5-307135, it is structured that a drying cauldron per se stands upright in normal times, and an upper portion of the drying cauldron is always opened, and also the drying cauldron can fall down only when the drying cauldron is cleaned. Heat accumulators are stored inside the drying cauldron and a lid plate is provided at the upper opening of the drying cauldron wherein the lid plate can be closed when the drying cauldron falls down. According to this structure, although the drying cauldron per se stands still and stands upright when night soil which is thrown in the drying cauldron is subjected to evaporating drying treatment, the drying cauldron falls down at the time of implementing cleaning treatment after night soil is evaporated so that dust remaining inside the drying cauldron can be discharged through the opening thereof. At the time of discharging dust, the lid plate (a plurality of small holes are bored on its surface) automatically closes the opening of the drying cauldron to prevent the heat accumulators from being dropped off, so that dust alone can be discharged from the small hole of the lid plate to a dust dish, thereby simplifying the structure of the night soil treatment apparatus.

Further, according to the invention disclosed in Japanese Patent Application No. 5-314445, it is structured that a drying cauldron is rotatably supported and an output of a motor meshes with a periphery of the drying cauldron, and a plurality of spherical heat accumulators are stored in the drying cauldron wherein three baffles are inserted from a lid plate which is positioned at the upper portion of the drying cauldron toward the interior of the drying cauldron. With this structure, the drying cauldron per se is rotated about a vertical axis line, which is from the drying cauldron of a conventional night soil treatment apparatus so that night soil can be heated by an electromagnetic wave from a high frequency coil. The heat accumulators stored in the drying cauldron are not rotated together with the drying cauldron by the baffles but rotated on the bottom of the drying cauldron so that night soil can be stirred and heated. With this structure, since it is not necessary to rotate the stirring blade inside the drying cauldron, the entire height of the night soil treatment apparatus can be lowered. Further, in the structure for rotating the stirring blade, it has an excellent advantage to prevent foreign objects other than night soil (e.g. a ball point pen, clothing, a watch and so forth) which are thrown in the drying cauldron from being tangled with a rotating blade.

Further, according to the invention disclosed in Japanese Patent Application No. 6-151631, it is likewise structured that a drying cauldron is rotatably supported and an output of a motor meshes with a periphery of the drying cauldron, and a plurality of spherical heat accumulators are stored in the drying cauldron wherein one baffle is inserted from a lid plate positioned at the upper portion of the drying cauldron toward the interior of the drying cauldron. Even with this structure, the drying cauldron per se is rotated about a vertical axis line so that night soil can be heated by an electromagnetic wave from a high frequency coil. According to this invention, the baffle is formed of one piece fixed to a suction pipe, and the heat accumulators are not engaged in a plurality of baffles so that the heat accumulators can be reliably rotated by the rotation of the drying cauldron.

According to these newly proposed night soil treatment apparatus, excreted night soil can be heated while it is kept airtight inside the drying cauldron and it can be mixed when the stirring blade and the drying cauldron are rotated. The temperature of heated night soil is increased uniformly as a whole, and moisture which is a major component is evaporated to be converted into vapor to be emitted to the atmosphere. Evaporating speed of night soil is increased by heating and mixing thereof so that treating time of night soil can be reduced. Further, vapor dispersed in the atmosphere is rendered odorless by a catalyst and so forth, and hence even in a place where houses are crowded or many people gather, an offensive odor is not emitted, which is preferable in view of an environment protection.

It is very hygienic to evaporate and emit night soil in an airtight evaporation container, and an operation can be systematized so that it does not give a burden to an operator at the time of maintaining and so forth. However, most of the conventional night soil treatment apparatus has a structure that the drying cauldron is fixed and the stirring blade is rotated in the drying cauldron, thereby mixing night soil. According to this mechanism, if foreign objects other than night soil which cannot be evaporated is thrown in the drying cauldron, the rotating stirring blade and these foreign objects are tangled with each other, which becomes frequently a cause of a trouble. For example, there is a case where a metallic ball point pen, a belt, clothing and so forth are thrown in a toilet stool owing to careless miss of a user. When these foreign objects thrown in the drying cauldron are bitten between the stirring blade and drying cauldron, there occurs phenomena that the rotation of the stirring blade is stopped and the stirring blade and the drying cauldron are abraded.

Further, if the stirring blade is housed in the drying cauldron, a limited inner space of the drying cauldron is narrowed by the mechanism, which becomes a primary factor of causing the inner space of the drying cauldron not to be effectively utilized.

To solve such a drawback, according to the inventions disclosed in Japanese Patent Application No. 5-314445 and No. 6-151631 are structured to rotate the drying cauldron per se, thereby devising that night soil can be mixed without using the stirring blade. However, even with this mechanism, the drying cauldron has to be rotatably supported, while it is not moved vertically and supported in the manner that the central axis is not deviated. If the rotating drying cauldron is moved vertically, and the rotary shaft performs eccentric motion, the opening end of the drying cauldron is moved away from a lid plate so that air-tightness of the drying cauldron cannot be kept.

If the rotary operation of the drying cauldron is to be regulated as mentioned above, a regulation mechanism (a roller and so forth) in two directions of a vertical direction and a rotating direction is needed, and this regulation mechanism becomes complex necessarily. Further, if the regulation mechanism becomes complex, it takes time and effort in an operating step of assembling the drying cauldron, and also takes time and effort for removing the drying cauldron so as to inspect or maintain the night soil treatment apparatus.

Still further, dust remaining in the drying cauldron after night soil is evaporated and dried has to be removed so as to continuously implement treatment of night soil, but it was difficult to control the insertion of a suction pipe for cleaning into the drying cauldron by a given length. It has been conventionally controlled by detecting the amount of the movement of the suction pipe so as to vertically move the suction pipe by an appropriate length, whereby the mechanism for moving the suction pipe and a mechanism of controlling the suction pipe have been complex.

Accordingly, the inventor of this application filed the application of the night soil treatment apparatus which improved the foregoing drawbacks by Japanese Patent Application No. 6-219457. The newly proposed night soil treatment apparatus is characterized in that a rotation holding means is provided on the upper portion of a counter, and an evaporation container is suspended from the lower portion of the rotation holding means, and an electromagnetic heating means is provided on the lower surface of the evaporation container with a spaced interval. With the proposed structure, since the evaporation container is rotated while it is suspended, it is characterized in that any supporting means is not necessary to be provided at the low portion and the side surface thereof so that the mechanism for holding the evaporation container is very simplified.

Further, with this structure, only the evaporation container can be removed from the lower portion of the rotation holding means so that the interior of the evaporation container can be easily exposed to the outside so as to implement regular inspection and maintenance operation. Although it is possible to remove a covering means for covering the upper portion of the evaporation container to observe the interior of the evaporation container from above, various pipes and apparatuses connected to the covering means, respectively, have to be removed, so that steps involved in removing operation becomes very complex.

As mentioned above, in order to throw night soil in the evaporation container to treat night soil by heating and evaporating every evaporation container, it is necessary to provide a mechanism for stirring night soil so as to uniformly increase the temperature of night soil. There has been employed either of a structure to stir night soil by rotating a stirring blade at a center of the interior of the evaporation container or a structure to stir night soil by rotating the evaporation container while the stirring blade is fixed so as not to be rotated, respectively, for implementing stirring night soil. According to the mechanism for rotating stirring blade while the evaporation container is fixed (e.g. disclosed in Japanese Patent Application No. 63-190857 and so forth) of these structures, it is characterized in that a mechanism for journaling the stirring blade becomes simple, and the evaporation container can be firmly supported. However, since this structure has a mechanism to rotate the stirring blade at the portion close to the bottom of the evaporation container, dust remaining in the evaporation container is hardly collected after night soil is evaporated. This is caused by the fact that a pipe for sucking dust cannot be installed on a portion close to the bottom of the evaporation container due to the stirring blade, and dust has to be sucked from the portion close to the lower end of a rotary shaft for supporting the stirring blade.

Further, according to the mechanism for rotating the evaporation container and fixing the stirring blade (e.g. Japanese Patent Application No. 6-151631 and so forth), there is adopted a mechanism wherein the evaporation container is airtightly and rotatably provided underneath a flat top plate, and a pipe provided with a baffle which is directed downward from the top plate toward the evaporation container. With this mechanism, the baffle is rotated relatively at the portion close to the bottom of the evaporation container when the evaporation container is rotated so that night soil which is rotated together with the evaporation container can be stirred. With this mechanism, since the holding pipe approaches the bottom of the evaporation container and it is positioned while it is deviated from the center of the evaporation container, it is characterized in that dust remaining in the bottom of the evaporation container is uniformly sucked, thereby reliably cleaning the evaporation container.

However, with this mechanism, since the evaporation container has to be held so as to be rotated, there are drawbacks in this mechanism that the mechanism for holding the evaporation container becomes complex, and the heavy evaporation container cannot be suspended. Further, since a gap between the top plate and the rotating evaporation container has to be held to be airtight and rotatable, there is a shortcoming that airtightness of the joining portion thereof has to be enhanced. Since the upper end opening of the evaporation container is relatively wide, a seal member having a large diameter and so forth have to be used so as to rotatably hold the evaporation container while the opening is kept in airtightness, there is a drawback that manufacturing cost becomes high.

Further, Japanese Patent Application No. 9-180755 is structured that it comprises a night soil tank capable of storing night soil, a heat resistant closed container for storing night soil, a heating means for heating the container to evaporate night soil, a night soil hopper having a capacity equivalent to the quantity of a load of night soil in the container to be treated, a closing valve intervened between the container and night soil hopper, a night soil pipe for causing night soil to flow and a night soil pump connected between the night soil tank and the night soil hopper in the midway of the night soil pipe. Further, the night soil pump has a function to crush sucked night soil therein, and spherical heat accumulators and stirring means for stirring night soil stored in the container and the heat accumulators are housed in the container.

With this structure, night soil in the night soil tank is sucked by the night soil pump to be stored in the night soil hopper, and a load of night soil to be subjected to drying treatment is thrown in the night soil hopper by opening the closing valve, thereby forming a batch type night soil treatment apparatus in which a tank for storing night soil and a container for implementing heating treatment are separated, wherein night soil is always circulated so that night soil stored in the tank is not solidified, and an odor generated in the tank is sucked and then subjected to oxidation and reduction by catalysts, so as to be treated to become odorless, thereby preventing an offensive odor from being generated in the night soil treatment apparatus.

As mentioned in detail above, there has been conventionally utilized a treatment apparatus which is considered to be hygienic, wherein night soil is introduced into the airtight container, and the container is heated, thereby evaporating moisture which is a major component of night soil to so as to be dispersed outside as vapor. With such a night soil treatment apparatus, feces and urine are directly thrown in the container so as to be heated from the toilet stool, and a gap between the toilet stool and the container is closed to thereby close the container, thereby preventing an odor of feces and urine from being leaked outside.

With the conventional structure in which the toilet stool and container are directly connected to each other, a capacity to treat night soil within a predetermined time is decided by an interior capacity of a container for storing night soil therein. That is, if night soil having a quantity exceeding a treating capacity of the container is introduced into the container, the toilet stool can not be used so that non-use state is maintained. For example, if many people use the toilet stool to excrete night soil within a short period of time, night soil is directly thrown in the container so that night soil overflows from the container to become overflowing state, which cannot be treated. Accordingly, it has been employed a system wherein if night soil having a quantity exceeding a treating capacity of the container is thrown in the container, use of the toilet stool has to be once stopped so that entire night soil filled in container is subjected to drying treatment, thereafter the use of the toilet stool can be allowed.

As mentioned above, since there has been employed a system to suspend or allow the use of the toilet stool corresponding to the treating capacity of the container, the conventional night soil treatment apparatus is limited in quantity of night soil to be subjected to drying treatment in a predetermined period of time, so that it cannot cope with excretion of many people in a short period of time. In order to treat night soil of many people, the capacity of the container for storing night soil is conventionally made large or a plurality of independent night soil treatment apparatuses are arranged in parallel with one another, thereby coping with night soil to be subjected to drying treatment within a scope of treating capacity respectively. However, according to the conventional night soil treatment apparatus, the capacity and the number of the container have to be set in conformity with the maximum number of people who use the toilet stool, so that in the case where many people utilize the toilet stool (temporary toilet) in a short rest time at an event site, the toilet stool has to be installed in conformity with the maximum number of people who utilize the toilet stool, causing the toilet stool to be uneconomical.

Accordingly, there has been conceived a mechanism wherein a tank is connected to a toilet stool, and a night soil excreted in the toilet stool is first stored in the tank, then night soil stored in the tank is subsequently thrown in the container, thereby continuously subjecting night soil to an evaporating drying treatment (batch treatment apparatus). With such a mechanism, it is not necessary to set the capacity of the container to be so large, so that even if a utilizer temporarily and concentrically uses the toilet stool, the excreted night soil can be stored in a relatively large tank. Then, night soil in the tank is sequentially thrown in the container to be continuously treated, entire night soil can be treated while taking time. For example, even if the tank is filled with night soil, night soil can be continuously treated at midnight so that even if the capacity of the container is small, it can treat night soil. Accordingly, even if a container having a large capacity is not used, night soil can be treated so that a fixing cost becomes low, thereby enhancing an efficient operation.

However, the mechanism directly connecting between the night soil tank and toilet stool so as to implement a batch treatment has serious drawbacks. That is, if night soil stored in the tank is kept remaining in the tank, the surface of night soil contacts oxygen to be oxidized with time, and is solidified, and hence fluidity is deteriorated so that it becomes difficult to discharge night soil outside the tank. Further, since the toilet stool and tank are directly connected to each other, an odor generated from the surface of night soil is filled in an inner surface of a temporary toilet and so forth through a lower opening of the toilet stool so that an offensive odor floats which is unpleasant to the user. According to the structure wherein the tank for storing night soil is separated from the night soil treatment apparatus, thereby treating night soil sequentially in a batch system, the capacity of the container is not necessary to be enlarged but it becomes a cause of generation of an odor from the tank.

Accordingly, Japanese Patent Application No. 9-180755 discloses the batch type night soil treatment apparatus wherein the tank for storing night soil and the container for subjecting night soil to heating treatment are separated, wherein night soil is always circulated so that night soil stored in the tank is not solidified, and an odor generated in the tank is sucked and is subjected to oxidation and reduction by the catalysts to be treated odorless, thereby preventing an offensive odor being generated.

Meanwhile, according to a conventional apparatus, night soil per se remaining in the drying cauldron serving as a container for treatment is subjected to dust and is separated from air to be collected. Accordingly, And, an odor generated in piping for sucking dust remaining in the drying cauldron to clean the drying cauldron and for rendering an odor generated in the night soil tank to be odorless to be discharged outside.

However, such a mechanism is a means for collecting a residue which is still kept in a state of night soil. For example, the fact that night soil residue in the drying cauldron is detected by a residue sensor so that it is detected that night soil is stored inside the night soil tank. A signal detecting the existence of night soil is detected by the residue sensor so that the pump and selecting valve are operated by a pump control circuit, and a discharge pipe and a return pipe communicate with each other, thereby collecting night soil stored in the drying cauldron by the pump.

Meanwhile, night soil which has not been collected when the toilet stool is continuously used is slightly carbonized inside the drying cauldron, and carbide thereof is adhered onto the interior of the drying cauldron little by little and accumulated therein. A location where carbide is accumulated is a ceiling portion, a peripheral wall portion, a bottom portion and so forth of the interior of the drying cauldron.

Accordingly, if the interior of the drying cauldron is not cleaned periodically, carbide accumulated in the bottom of the cauldron becomes resistance to the rotation of the stirring blade, causing abnormal noise due to friction to occur or the stirring motor to be troubled.

Further, if the interior of the drying cauldron is not cleaned periodically, carbide accumulated in the bottom of the drying cauldron prevents heat transfer of the heater provided at the bottom of the cauldron. If the apparatus is structured to be controlled based on a cauldron bottom temperature and a heater temperature, the process proceeds in an incomplete treating state.

Further, if the interior of the drying cauldron is not cleaned periodically, carbide adhered onto the upper surface of the cauldron interior and a carbide collection piping inlet is moisturized and fixed together with condensation inside the cauldron interior, so that carbide is liable to be further adhered, and it becomes large when used frequently. As a result, the problem of clogging of piping and so forth occur.

To that end, in order to remove carbide, it is necessary to remove the drying cauldron or top plate periodically, thereby cleaning the interior of the drying cauldron. However, it requires a working by an operator to detach the top plate from the drying cauldron, a working to scrape carbide in the drying cauldron, a working for collecting the scraped carbide and so forth, thereby requiring cost and time involved in such troublesome working.

On the other hand, there occurs similar phenomena in catalysts in the deodorizing portion. That is, a deodorizing apparatus for extinguishing an odor discharged from the drying cauldron is provided inside a deodorizing case. The deodorizing apparatus, for example, stores catalysts having various shapes in the catalyst case and catches an odor component through discharge air which passes through the catalysts. However, foreign objects of fine particle contained in deodorizing component cannot be collected completely in its entire quantity so that it adheres onto the interior of the catalysts little by little as a foreign object and accumulated therein.

Accordingly, if the interior of the catalyst case is not cleaned periodically, the flow of an odor is prevented by the adhered foreign object so that an inherent deodorizing function cannot be maintained. Further, a working for taking out the catalysts for cleaning has to be implemented after a heater is sufficiently cooled, requiring a cooling time, while if a cleaning is implemented without confirming cooling thereof, there is a likelihood of occurrence of danger such as burn injury and so forth.

Further, the catalyst case is manufactured by a material of suitable roughness, which is neither too course nor so fine, fine foreign objects which can not be captured reach the catalyst section, and adhere to the surfaces of catalysts. Accordingly, it is necessary to take out the catalysts periodically to cleat it, however, the attachment and detachment of the catalysts which are filled in the catalyst case with no space therebetween are very difficult working. Further, there is a likelihood that the catalysts are broken by heating and vibration and the broken pieces are clogged in the catalysts.

### SUMMARY OF THE INVENTION

It is an object of the invention to provide a night soil drying treatment apparatus provided with an automatic washing function for removing adherents onto an interior of a drying cauldron by automatically washing the interior of the drying cauldron every time one cycle operation is completed.

It is another object of the invention to provide a night soil drying treatment apparatus provided with a cleaning function for automatically washing surfaces of metallic catalysts, thereby keeping the metallic catalysts clean every time one cycle of treatment of a drying cauldron is completed.

A cauldron interior cleaning mechanism of a night soil drying treatment apparatus according to the first aspect of the invention comprising a temporary toilet provided with a night soil tank capable of storing therein night soil and a toilet stool communicating with the night soil tank at its lower portion for causing night soil to flow down, and a night soil drying treatment mechanism comprised of a heat resistant closed drying cauldron for storing night soil therein, and a heating means for heating the drying cauldron to evaporate night soil, thereby subjecting night soil to drying treatment by evaporating night soil in the airtight drying cauldron, wherein a night soil pipe for causing night soil to fluid therein is connected between the night soil tank and the drying cauldron for causing night soil inside the night soil tank to be sucked up and thrown in the drying cauldron, and upon storing a load of night soil to be subjected to drying treatment in the drying cauldron, the drying cauldron is heated to evaporate night soil in the drying cauldron, and the heating and evaporating treatment are carried out by a set number to complete night soil drying treatment, characterized in that the cauldron interior cleaning mechanism is comprised of a washing mechanism for jetting water to the interior of the drying cauldron to wash the interior of the drying cauldron upon completion of night soil dying treatment, thereby removing adherents onto the interior of the drying cauldron.

The cauldron resistor cleaning mechanism according to the second aspect of the invention is characterized in that the washing mechanism comprises a water tank for washing the toilet stool, a washing water pipe for introducing water from the water tank into the drying cauldron, a high pressure water generation pump provided in the washing water pipe, and jet nozzles provided at the tip end of the washing water pipe for jetting high pressure water toward the interior of the drying cauldron.

The cauldron interior cleaning mechanism according to the third aspect of the invention is characterized in that the jet nozzles are provided on a stirring shaft rotating in the drying cauldron to jet washing water to the interior of the drying cauldron while the stirring shaft is rotated.

The cauldron interior cleaning mechanism according to the fourth aspect of the invention is characterized in that the washing pipe is divided into plural numbers through which washing water is jetted toward different positions of the interior of the drying cauldron by switching over valves, thereby individually washing the interior of the drying cauldron.

A catalyst cleaning mechanism of a night soil drying treatment apparatus according to the fifth aspect of the invention comprising a temporary toilet provided with a night soil tank capable of storing therein night soil and a toilet stool communicating with the night soil tank at its lower portion for causing night soil to flow down, and a night soil drying treatment mechanism comprised of a heat resistant closed drying cauldron for storing night soil therein, and a heating means for heating the drying cauldron to evaporate night soil, thereby subjecting night soil to drying treatment by evaporating night soil in the airtight drying cauldron, wherein a night soil pipe for causing night soil to fluid therein is connected between the night soil tank and the drying cauldron for causing night soil inside the night soil tank to be sucked up and thrown in the drying cauldron, wherein the drying cauldron is heated to evaporate night soil in the drying cauldron, said night soil drying treatment apparatus further comprising a discharge air mechanism for sucking air and moisture evaporated in the drying cauldron, a catalyst mechanism intervened between the drying cauldron and the discharge air mechanism for subjecting elements of odor to oxidation and reduction, and an odor pipe connected between an inlet side of the catalyst mechanism and an upper space of the night soil tank for causing air to fluid therebetween, and wherein air and moisture is discharged outward by the discharge air mechanism during an evaporating drying treatment in the drying cauldron, and elements of odor are subjected to oxidation and reduction by the catalyst mechanism in the middle of discharge of air and moisture, and air remaining in the upper spaces of the night soil tank, piping and drying cauldron is sucked by the odor pipe to be passed through the catalyst mechanism, thereby rendering the odor floating in the space of the night soil tank odorless, characterized in that the catalyst cleaning mechanism is comprised of a wind pressure cleaning mechanism for supplying compressed air to the catalyst mechanism upon completion of drying treatment to blow off adherents onto the surfaces of catalysts by a wind pressure.

The night soil drying treatment apparatus according to the sixth aspect of the invention is characterized in that the wind pressure cleaning mechanism comprises a compressor, and an air tank for storing therein compressed air supplied from the compressor and instantaneously jetting compressed air toward the catalyst mechanism when implementing cleaning treatment.

The night soil drying treatment apparatus according to the seventh aspect of the invention is characterized in that the wind pressure cleaning mechanism is set to implement cleaning treatment every time a load of night soil is treated in the drying cauldron.

### BRIEF DESCRIPTION OF THE DRAWINGS

FIG. 1 is a perspective view of an entire outward appearance of the night soil drying treatment apparatus of the invention as viewed from the slanting front thereof;

FIG. 2 is a perspective exploded view of a toilet stool, a water tank and a night soil treatment apparatus in the night soil drying treatment apparatus of the invention;

FIG. 3 is an exploded view of the toilet stool, night soil tank and so forth in the night soil drying treatment apparatus of the invention;

FIG. 4 is a view schematically showing the internal structure of the night soil treatment apparatus in the night soil drying treatment apparatus of the invention;

FIG. 5 is a perspective view of a housing box which is shown by cutting along the line J-J in FIG. 4 in order to indicate the arrangement of an internal mechanism of the night soil treatment apparatus in the night soil drying treatment apparatus of the invention;

FIG. 6 is a perspective view of a housing box which is shown by cutting along the line K-K in FIG. 4 in order to indicate the arrangement of an internal mechanism of the night soil treatment apparatus in the night soil drying treatment apparatus of the invention;

FIG. 7 is a longitudinal sectional view showing the structures of apparatuses of a washing mechanism in the night soil drying treatment apparatus of the invention;

FIG. 8 is a perspective exploded view of the apparatuses of the washing mechanism provided in the drying cauldron of the night soil treatment apparatus in the night soil drying treatment apparatus of the invention;

FIG. 9 is a perspective exploded view of a preheating section, a catalyst section and constituent elements of a catalyst cleaning mechanism of the night soil treatment apparatus in the night soil drying treatment apparatus of the invention;

FIG. 10 is a longitudinal sectional view of an outer cylinder of the preheating section of the night soil treatment apparatus taken along the central axis in the night soil drying treatment apparatus of the invention;

FIG. 11 is a perspective view showing exploded structures of the constituent members of the preheating section of the night soil treatment apparatus in the night soil drying treatment apparatus of the invention;

FIG. 12 is a longitudinal sectional view of the structure of the catalyst section in the night soil drying treatment apparatus of the invention;

FIG. 13 is a perspective view showing exploded structures of the constituent members of the catalyst section of the night soil treatment apparatus in the night soil drying treatment apparatus of the invention;

FIG. 14 is a perspective view showing a structure of a dust collection section of the night soil treatment apparatus in the night soil drying treatment apparatus of the invention;

FIG. 15 is schematic view of the structure of the connection of piping of the night soil treatment apparatus in the night soil drying treatment apparatus of the invention;

FIG. 16 is a schematic view of the structure of the connection of the entire piping of the night soil treatment apparatus in the night soil drying treatment apparatus of the invention;

FIG. 17 is a block diagram showing an electric system for controlling all operations in the night soil drying treatment apparatus of the invention;

FIG. 18 is a flow chart showing all steps for driving the night soil treatment apparatus in the night soil drying treatment apparatus of the invention;

FIG. 19 is a schematic view for explaining the operation of night soil drying treatment in the night soil drying treatment apparatus of the invention;

FIG. 20 is a flowchart showing a procedure of a dust collecting step in the night soil drying treatment apparatus;

FIG. 21 is a schematic view showing the structures of the apparatuses used in the dust collecting step in the night soil drying treatment apparatus of the invention;

FIG. 22 is a flowchart showing a procedure of a drying cauldron cleaning step in the night soil drying treatment apparatus of the invention;

FIG. 23 is a schematic view of the structures of the apparatuses used in the night soil drying treatment apparatus of the invention;

FIG. 24 is a flowchart showing a procedure of a catalyst washing step in the night soil drying treatment apparatus of the invention; and

FIG. 25 is a schematic view of the structures of the apparatuses used in the catalyst washing step in the night soil drying treatment apparatus of the invention.

### PREFERRED EMBODIMENT OF THE INVENTION

An embodiment of the invention is now described with reference to the attached drawings. According to the present embodiment, a night soil drying treatment apparatus is described with reference to a mechanism capable of treating feces and urine. However, the invention is not limited to this embodiment, and it is needless to say that the invention can achieve the same object even if the invention is limited to a mechanism which is intended for subjecting urine alone to drying treatment. The present embodiment is described in the case where it is applied to a temporary toilet system, wherein the mechanism of the present embodiment is separated into a movable temporary toilet 1 and a night soil treatment apparatus 11 for heating and evaporating night soil wherein both the temporary toilet 1 and the night soil treatment apparatus 11 are loaded on a truck and so forth or carried by the truck and so forth so as to be temporarily used in an event site, a river bed, a disaster area caused by a wind and snow, a flood, an earth quake and so forth.

Entire structure of the night soil drying treatment apparatus:

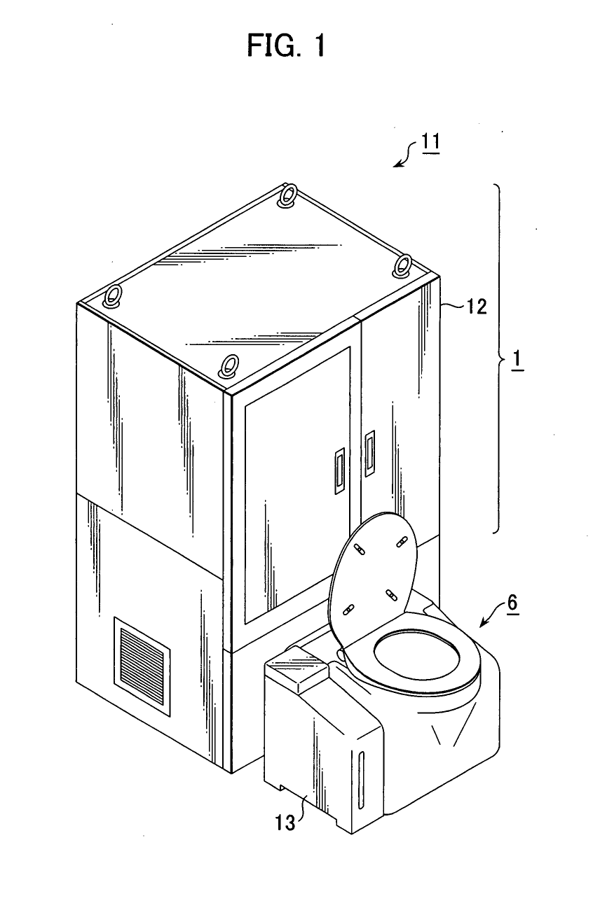

FIG. 1 is a perspective view of an entire outward appearance of the night soil drying treatment apparatus according to the embodiment as viewed from the slanting front thereof. The night soil drying treatment apparatus is mainly divided into two components. That is, it comprises a temporary toilet 1 and a night soil treatment apparatus 11. The temporary toilet 1 has a well-known shape which is housed in a house, not shown, and has been conventionally used in a construction site, an event site, and so forth. Respective apparatuses of the night soil treatment apparatus 11 are housed in a substantially rectangular parallelepiped housing box 12 for housing the apparatuses therein.

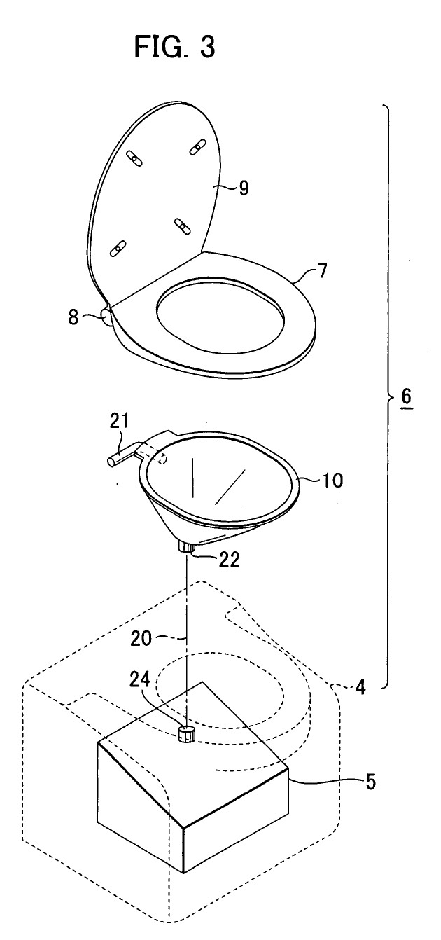

The structure of respective apparatuses inside the housing box 12 will be described in detail with reference to FIG. 3 to FIG. 5, but they are roughly described herein. There are housed in the housing box 12 a quantitative tank for use in batch treatment and for temporary housing night soil therein by sucking night soil by a fixed quantity, and a drying cauldron formed of an airtight container serving as a drying portion for subjecting night soil to drying treatment while drying night soil from the quantitative tank. The night soil Will be evaporated in an airtight state while it is sucked toward the drying cauldron. Upon completion of subjecting a set full quantity of night soil to drying treatment, water is jetted toward the interior of the drying cauldron to wash the interior of the drying cauldron to remove the adherents onto the interior of the drying cauldron.

Upon completion of water washing treatment, the drying cauldron is further heated to evaporate moisture. Vapor and air inside the drying cauldron generated at the time of heating are introduced into to a catalyst mechanism and they are deodorized and discharged outside. Foreign objects are adhered onto catalysts housed in the catalyst mechanism. Accordingly, the surfaces of catalysts have a cleaning function for automatically flushing thereof every time one cycle drying treatment is completed in the drying cauldron, thereby keeping the catalysts clean.

An outer frame, not shown, for covering a toilet stool 6 of the temporary toilet 1 is formed of, for example, plastics and reinforced synthetic resins, and has a cubic box shape as a whole. A bottom portion of the temporary toilet 1 is formed of a base table to be installed on the ground, a bed of a truck and a bed of a trailer, and it is square in its flat shape, low in height and flat cubic. A space through which a user enters is formed on the upper surface of the base table, and a house having a house shape, not shown, provided with a roof on the top portion thereof is placed thereon. The front of the house is provided with doors attached thereto through which the user enters the house by pulling the doors by the user. A night soil tank for first storing feces and urine is fixed to the upper portion of the base table inside the temporary toilet 1. The night soil tank will be explained later with reference to FIG. 3 and succeeding figures.

The western style toilet stool 6 is fixed to the center of the upper surface of the night soil tank so that the user of the temporary toilet 1 can excrete night soil toward the toilet stool 6. The toilet stool 6 is opened at the bottom surface and communicates with an inner space of the night soil tank. There are provided a water tank 13 serving as a water washing apparatus and an operation portion for use in water washing operation, not shown, and so forth at the side, e.g. at the right side (shown in left side of the toilet stool 6 in FIG. 1) in a state where the utilizer sits on the toilet stool 6. Meanwhile, the night soil treatment apparatus 11 is installed inside the rectangular parallelepiped housing box 12, and an airtight mechanism, a heating mechanism, a washing mechanism which is automatically operated and so forth are housed inside the housing box 12, and they have a function to treat night soil by evaporating it, and implement an interior cleaning and so forth.

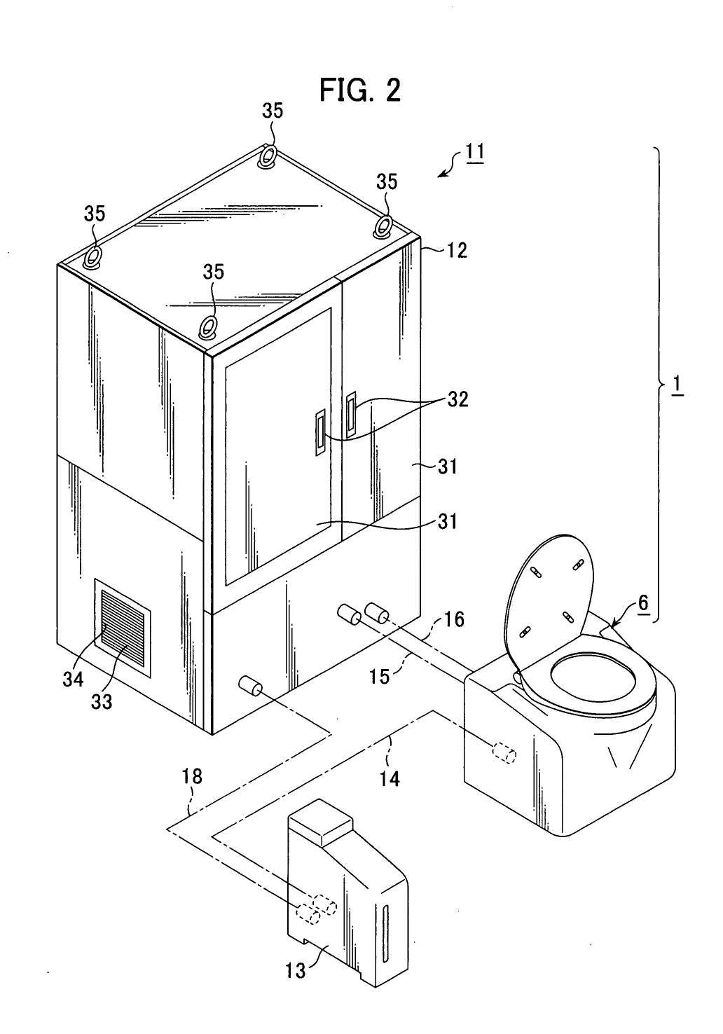

FIG. 2 shows that the toilet stool 6, water tank 13 and the night soil treatment apparatus 11 shown in FIG. 1 are exploded, and also shows the structures of piping and entire night soil treatment.

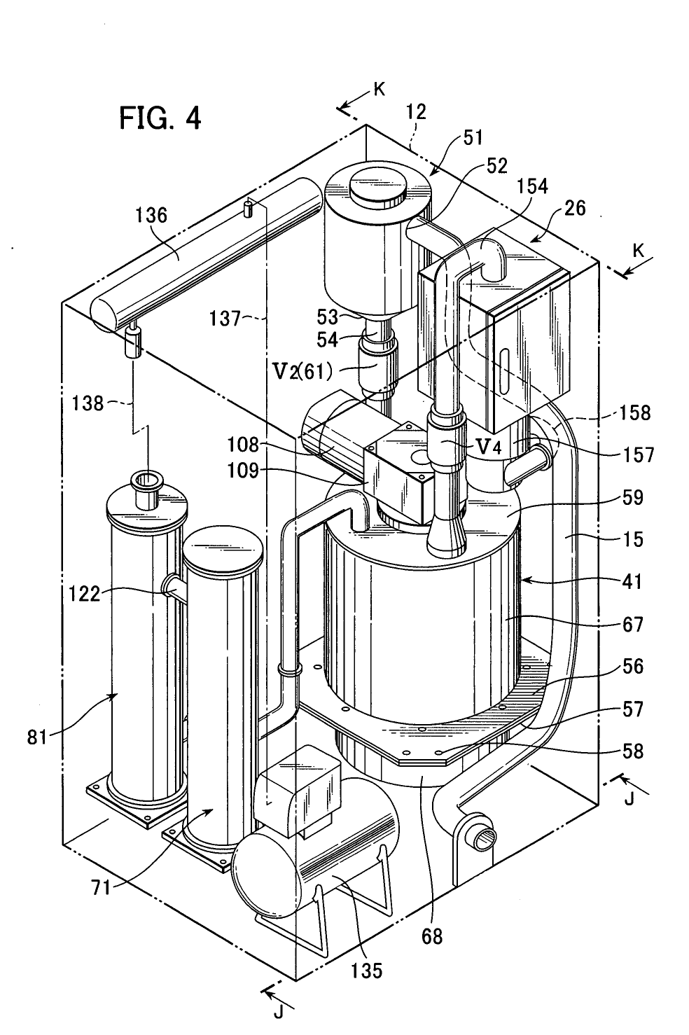

As showing FIG. 2, the toilet stool 6 and water tank 13 disposed at the side of the toilet stool 6 are connected to each other by a flushing water pipe 14. When water is supplied from the water tank 13 to the toilet stool 6 via the water washing pipe 14, night soil excreted inside the toilet stool 6 is washed out to clean the toilet stool 6. A night soil transfer pipe 15 extended toward the back of the toilet stool 6 is provided in the toilet stool 6, and it transfers night soil to the quantitative tank (shown in FIG. 4 and succeeding figures) provided inside the night soil treatment apparatus 11 from the night soil tank provided under the toilet stool 6 (shown in FIG. 3 and succeeding figures). An odor discharge pipe 16 is provided at the rear portion of the toilet stool 6, and it is connected to a catalyst section via the night soil tank, a discharge air pipe provided in the night soil drying treatment apparatus, and so forth, so as to discharge air from which an odor is eliminated at the catalyst section toward the open air.

An end of a water washing pipe 18 for supplying washing water toward the housing box 12 of the night soil treatment apparatus 11 is connected to the water tank 13, and the other end of the water washing pipe 18 is connected to an interior of the drying cauldron provided inside the night soil treatment apparatus. Inside the drying cauldron, night soil supplied thereto is subjected to drying treatment by heating, described later, and the foreign objects remained therein are fed to a collection container to be collected. Water supplied from the water washing pipe 18 is jetted toward the interior of the drying cauldron so as to clean the foreign objects remained in the drying cauldron, which is implemented after drying treatment so as to implement washing treatment in the drying cauldron.

As shown in the left side of FIG. 2, the night soil transfer pipe 15, odor discharge pipe 16 and water washing pipe 18 are connected to the front surface of the housing box 12, namely, at the lower portion of the side surface facing the toilet stool 6 disposed at the right front side in FIG. 2. A pair of doors 31, 31 to be openable and closable at the right and left are provided in the housing box 12 and positioned over the night soil transfer pipe 15, odor discharge pipe 16 and water washing pipe 18 so that an operator opens and closes the doors 31 to the right and left to enter the housing box 12 when implementing maintenance inspection and so forth. A necessary air duct is provided at the side surface of the housing box 12. For example, in the case of FIG. 2, an air duct 34 provided with a louver 33 is shown at the lower portion of the side surface of the housing box 12 at the left front side thereof. Hoisting hooks 35 are provided at four corners on the top surface of the housing box 12 and they are hoisted, e.g. by a wire and so forth by use of a crane of a crane truck, so that the entire housing box 12 is moved to be installed and removed.

FIG. 3 is a view showing structures of the toilet stool 6, night soil tank and so forth in an exploded manner. The toilet stool 6 comprises a toilet seat 7 having various shapes such as a substantially O-shape as shown in FIG. 3, a U-shape, not shown, and a toilet lid 9 to be openable vertically which is attached to the rear portion of the toilet seat 7 via a hinge 8. A conical toilet bowl 10 which is wide open at the upper surface is provided under the toilet seat 7. A coupling 21 for water supply pipe connected to the water washing pipe 14, shown in FIG. 2, is provided at the back side of the upper end of the toilet bowl 10, namely at the position under the hinge 8 side for supporting the toilet lid 9. A night soil discharge port 22 through which night soil is discharged is opened at the lower central portion of the toilet bowl 10, and a night soil pipe 20 directing downward is substantially vertically disposed in the night soil discharge port 22. The night soil pipe 20 has a valve, not shown, for shutting the discharge of an odor upward. This valve is opened when discharging flushing water at the time of evacuating but it is closed in a normal time.

The toilet seat 7 and toilet bowl 10 having such a structure is supported by the box-shaped toilet table 4, and a night soil tank 5 is provided under the interior of the toilet table 4. The night soil tank 5 is, e.g. a box-shaped tank, and a night soil introduction port 24 is provided on the night soil tank 5 at the upper central portion. The night soil pipe 20 directing downward from the toilet bowl 10 is connected to the night soil introduction port 24, and excreted night soil is to be stored inside the night soil tank 5 via the night soil pipe 20. A full water detection float sensor, described later, is provided in the night soil tank 5 so as to detect whether the quantity of night soil to be contained is in a drying treatment level or not. A night soil discharge mechanism is provided in the night soil tank 5 for pressurizing the upper surface side of night soil to be stored in the night soil tank 5 by an air pressure, thereby discharging night soil outward from the night soil discharge port 22 provided at the lower portion of the night soil tank 5.

Outline of the structure inside the night soil treatment apparatus 11:

FIG. 4 is a view schematically showing the internal structure of the night soil treatment apparatus 11.

Meanwhile, FIG. 4 is a perspective view showing a state where the interior of the housing box 12 is viewed in the same angle of the field of view as those of FIG. 1 and FIG. 2. The housing box 12 of the night soil treatment apparatus 11 has a rectangular parallelepiped housing box in an outer shape as shown in FIG. 1, and it is formed of an iron sheet. The inner constituent elements of the temporary toilet 1 are surrounded by the housing box 12 (by imaginary line) and various mechanisms are assembled in the night soil treatment apparatus 11 (FIG. 4 shows a state where the peripheral iron sheet is removed).

As shown in FIG. 4, a quantitative tank 51 capable of temporarily storing a given quantity of night soil by sucking night soil from the night soil tank 5 shown in FIG. 3 is provided in the housing box 12 at the upper position of the right innermost portion of the housing box 12. The quantitative tank 51 is longitudinally cylindrical, and a night soil suction port 52 for sucking night soil is provided on the upper portion of the quantitative tank 51. A terminal end of the night soil transfer pipe 15, which stands upright from the lower portion of the housing box 12 at the front side through a space inside the housing box 12, is connected to the night soil suction port 52 of the quantitative tank 51. The night soil transfer pipe 15 shown in FIG. 2 is connected to the opening end of the night soil transfer pipe 15 at its lower end side to form one pipe. The lower portion of the quantitative tank 51 forms a funnel-shaped (reverse conical) night soil discharged part 53, and a night soil drying transfer pipe 54 for transferring night soil to the drying cauldron to implement drying treatment is provided on the lower end of the night soil discharged part 53 at the central position thereof. A closing valve 61 formed of an electromagnetic valve for controlling the transfer of night soil which is opened at the time of transferring night soil is provided on the night soil drying transfer pipe 54 at the upper position thereof. A given quantitative detection section for detecting that a given quantity of night soil is stored, described later, is provided in the quantitative tank 51. Next, a drying cauldron 41 for implementing night soil drying treatment is disposed at the right lower portion of the housing box 12 located substantially at the central position in a front-to-back direction. The drying cauldron 41 stores a given quantity of night soil which is supplied from the quantitative tank 51 via the night soil drying transfer pipe 54 and implements drying treatment. The drying cauldron 41 is longitudinally cylindrical as a whole, and comprises an upper cauldron section 67 which is opened at the lower surface and a lower cauldron section 68 which is opened at the upper surface, which are vertically connected to each other, thereby forming a space for use in treatment which is kept airtight inside thereof. That is, a square upper flange 56 which is cut at four corners is provided on the lower end periphery of the cylindrical portion of the upper cauldron section 67, and a square flange 57 which is cut at four corners like the square upper flange 56 of the upper cauldron section 67 is provided on the upper end periphery of the cylindrical portion of the lower cauldron section 68, wherein both flanges 56 and 57 are connected to each other by bolts 58 to structure the drying cauldron 41 which is kept airtight inside as a whole.

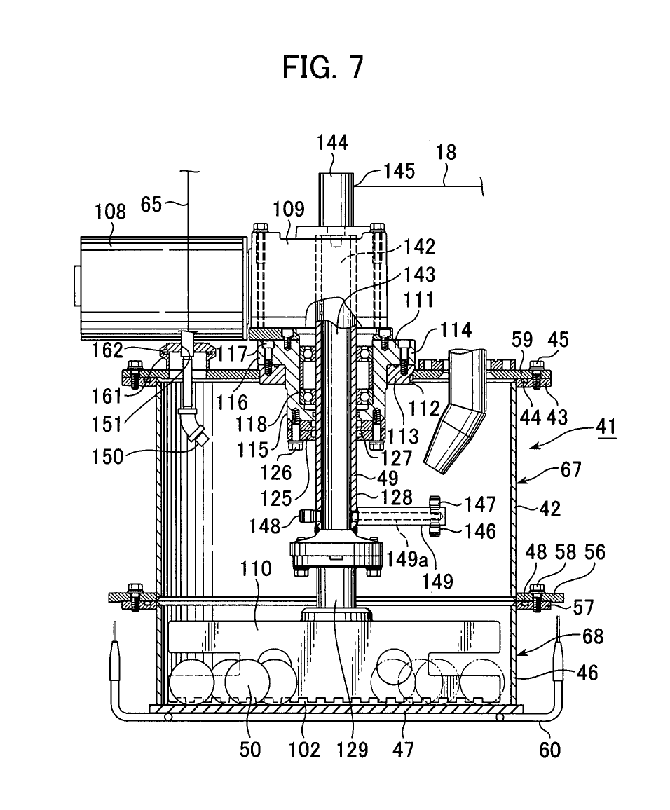

A top plate of the upper surface of the upper cauldron section 67 forms a detachable cauldron lid 59, and the cauldron lid 59 is fixed to an opening edge portion of the upper cauldron section 67 by bolts and so forth. The cauldron lid 59 has a pipe insertion hole which is in a hidden state in FIG. 4, and the night soil drying transfer pipe 54 for transferring night soil from the quantitative tank 51 is inserted into the pipe insertion hole. A given quantity of night soil which is stored in the quantitative tank 51 is to be sucked in the drying cauldron 41 via night soil drying transfer pipe 54 when implementing drying treatment. A motor 108 and a gear box 109 serving as a driving section of a stirring mechanism for stirring night soil which is transferred in the cauldron when implementing drying treatment are provided at the central position of the cauldron lid 59. A heater, described later, serving as a heating mechanism for heating the interior of the drying cauldron 41 when implementing drying treatment is provided in the lower cauldron section 68. Further, a drying cauldron washing mechanism for flushing the interior of the cauldron after implementing drying treatment is provided inside the interior of the drying cauldron 41. The drying cauldron washing mechanism comprises, described later, the same common components as those of the stirring mechanism and so forth. A mechanism inside the cauldron will be described in detail with reference to FIG. 7 and succeeding figures.

A box-shaped dust collection section 26 for separating dust such as carbide and so forth which is a solid material and generated upon implementing drying treatment in the drying cauldron 41 is disposed at the upper position of the drying cauldron 41 at the right side surface of the housing box 12. The dust collection section 26 has inside thereof a collection bag for collecting the dust and a suction device 157 such as dust sucking fan at the lower portion. The collection bag and dust sucking fan communicate with each other via an introduction pipe 154 for sucking the dust and a discharge air pipe 158 for returning air, wherein dust is captured from air which is circulating between the introduction pipe 154, discharge air pipe 58 and drying cauldron 41, and it is stored in the collection bag. Closing valves each formed of an electromagnetic valve are provided in the introduction pipe 154 and discharge air pipe 158. Although it is omitted in FIG. 4, an electromagnetic valve serving as a closing valve is also provided in a dust sucking pipe.

A preheating section 71 for heating discharge air while sucking it and a catalyst section 81 for adsorbing and removing preheated discharge air by the catalysts are disposed at the left side surface of the housing box 12 of the night soil treatment apparatus 11 for sucking elements of odor contained in discharge air from the quantitative tank 51, drying cauldron 41 and so forth. The preheating section 71 and catalyst section 81 are arranged in parallel with each other to form a pair of tubular structures. First, the preheating section 71 is connected to a discharge air piping 121 which is connected to the drying cauldron 41, so that the discharge air after treated in the drying cauldron 41 is introduced into to the preheating section 71 via the discharge air piping 121. The discharge air piping 121 has a closing valve formed of an electromagnetic valve, not shown in FIG. 4. Next, the catalyst section 81 is connected to the preheating section 71 via a communication pipe 122 so that foreign objects contained in discharge air are adhered onto the catalysts housed in the catalyst section 81 and removed. Discharge air that becomes clean while the foreign objects are removed by adhering onto the catalysts is discharged through an outer discharge air pipe, not shown, disposed at the back surface of the housing box 12.

A compressor 135 for generating air pressure to be utilized in the transfer of night soil, supply of air, cleaning of the catalysts and so forth is disposed at the front surface left side of the housing box 12. An air tank 136 is disposed in the housing box 12 at the upper portion of the back surface left side of the housing box 12. The air tank 136 is connected to the compressor 135 via a compressed air pipe 137 and temporarily stores a compressed air generated in the compressor 135. Accordingly, the air tank 136 is connected to the catalyst section 81 via a discharge pipe 138 serving as a discharge piping for jetting compressed air, and it instantaneously jets compressed air toward the catalyst section 81 when washing the catalysts, and removes the foreign objects adhered onto the catalysts by a wind pressure.

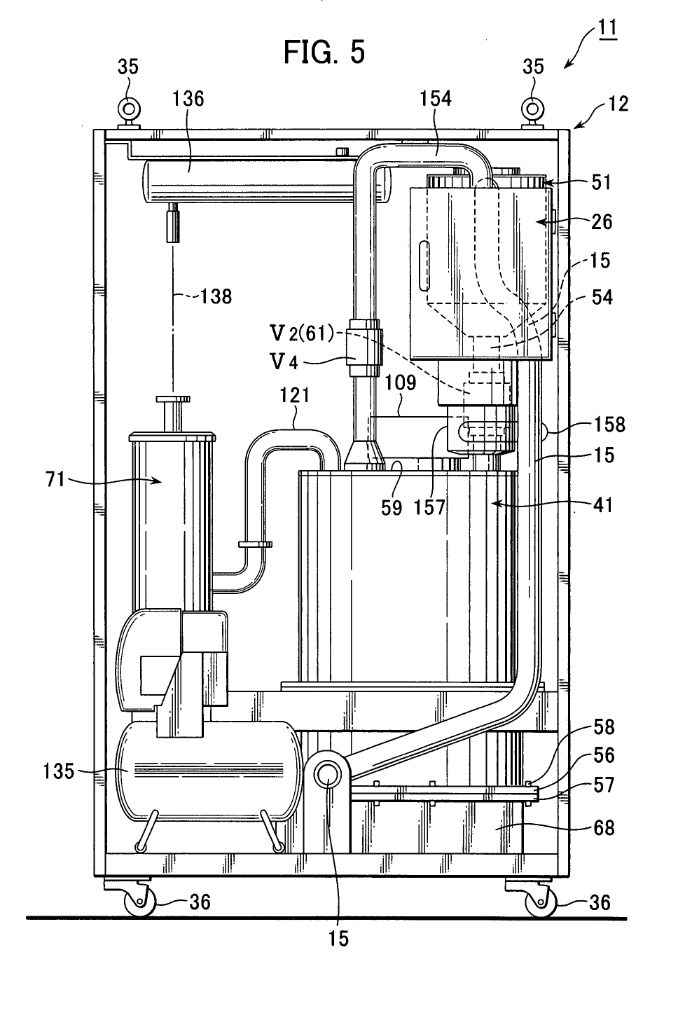

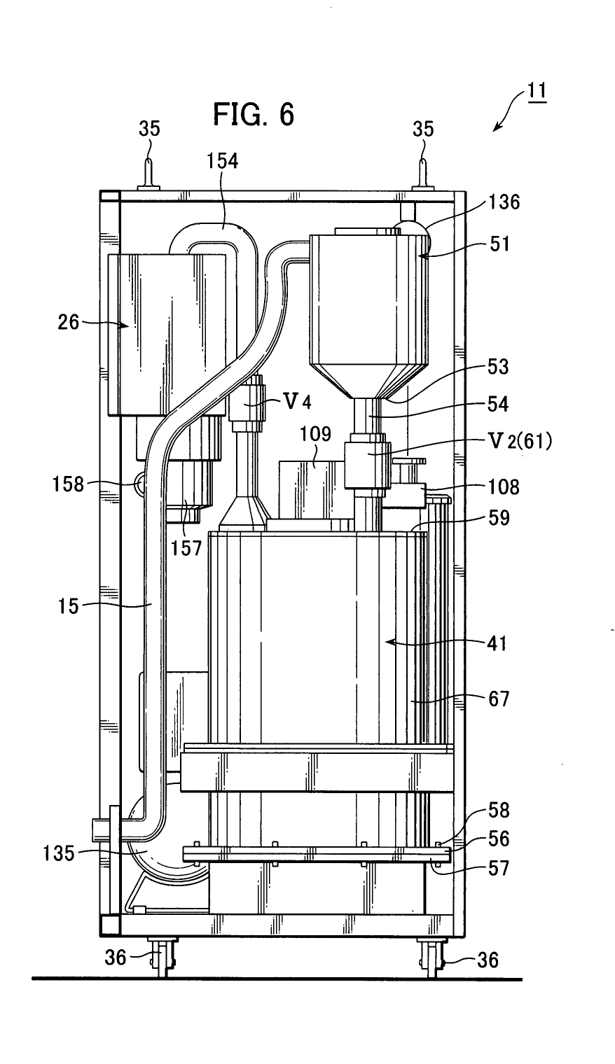

Next, FIG. 5 shows an internal structure of the housing box 12 of the night soil treatment apparatus 11 shown in FIG. 4 by viewing arrangement of various apparatuses stored in the housing box 12 from the front part (in a state as viewed from the left front side in FIG. 4) while cutting the housing box 12 along line J-J in FIG. 4. Further, FIG. 6 shows an internal structure of the housing box 12 of the night soil treatment apparatus 11 shown in FIG. 4 by viewing arrangement of various apparatuses stored in the housing box 12 from the front part (in a state sown from the right front side in FIG. 4) while cutting the housing box 12 along line K-K in FIG. 4.

As shown in FIG. 5 and FIG. 6, wheels 36 for moving the housing box 12 are provided at the lower end portion of the housing box 12 and the hoisting hooks 35 are provided at the upper end portion thereof. The compressor 135 is disposed at the lower portion of the housing box 12 and the preheating section 71 is disposed adjacent to the compressor 135. The air tank 136 is disposed over the preheating section 71. The night soil introduction section of the night soil transfer pipe 15 shown in FIG. 4 is disposed at the lowest portion of the housing box 12, and the night soil transfer pipe 15 stands upward, and also stands upward along the inner surface of the housing box 12, then it extends to the innermost side to be connected to the quantitative tank 51.

The night soil transfer pipe 15 for discharging night soil to the drying cauldron 41 is provided at the lower end central position of the quantitative tank 51 and it has the closing valve 61 (v2). The drying cauldron 41 is disposed at the lower portion of the interior of the housing box 12 and a given quantity of night soil is supplied from the quantitative tank 51 to the night soil transfer pipe 15 at the time of treating night soil. The motor 108 and gear box 109 are provided at the central position of the cauldron lid 59 of the quantitative tank 51. The box-shaped dust collection section 26 is disposed at the upper position of the drying cauldron 41 and the suction device 157 having the dust suction fan is provided at the lower portion of the dust collection section 26. Although the preheating section 71 inside the housing box 12 is illustrated at the left side of FIG. 5, the catalyst section 81 is rendered in a state to be hidden in FIG. 5. Further, the discharge air piping 121 for connecting the preheating section 71 and the drying cauldron 41 are illustrated in FIG. 5. Still further, the compressor 135 and air tank 136 disposed inside the housing box 12 are illustrated.

Structure of the drying section:

FIG. 7 shows a structure of the drying cauldron 41 serving as a main member of the drying section. The same figure shows the interior of the drying cauldron 41 as a longitudinal sectional view. The drying cauldron 41 sucks night soil supplied from the night soil tank 5 via the night soil transfer pipe 15 (omitted in FIG. 7) as described above and subjects night soil to drying treatment, and the drying cauldron 41 is formed of a metallic material such as heat resistant stainless steel, high tension iron steel and so forth. The drying cauldron 41 comprises the upper cauldron section 67 which is opened at the lower surface and the lower cauldron section 68 which is opened at the upper surface, wherein these openings are opposed to and connected to each other. First, the upper cauldron section 67 is structured to have a body part by a cylindrical body 42 which is opened at the upper and lower surfaces. An upper flange 43 is horizontally welded to the upper end outer peripheral side of the cylindrical body 42, and the circular cauldron lid 59 is joined to the flange 43 by bolts 45. A ring-shaped packing 44 is intervened between the joined portions of the flange 43 and cauldron lid 59, whereby the night soil drying transfer pipe 54 and cauldron lid 59 are airtightly sealed. A substantially square joining flange (square shaped upper flange) 56 having one side which is larger than an outer diameter of the upper cauldron section 67 is joined to the lower end outer peripheral edge side of the cylindrical body 42 of the upper cauldron section 67 by welding. Four corners of the joining flange 56 are cut respectively to form a compact structure. Accordingly, the upper cauldron section 67 is closed at the upper end side of the longitudinal cylinder, and is opened at the lower end side, while the joining flange 56 is provided at the lower end of the upper cauldron section 67.

Meanwhile, the lower cauldron section 68 forms a main portion by a cylindrical body 46 having the same diameter and thickness as the cylindrical body of the upper cauldron section 67, and it is opened at the upper and lower surfaces. The axial length (height) of the cylindrical body 46 of the lower cauldron section 68 is shorter than the cylindrical body 42 of the upper cauldron section 67, for example, it is one half or one third thereof. As a result, when the drying cauldron 41 is installed, for example, on the earth and so forth, a compression strength of the lower cauldron section 68 in a vertical direction is high, and hence it can be stably installed. A bottom plate 47 is welded to the lower end of the cylindrical body 46 of the lower cauldron section 68 while the flange 57 is joined to the upper end outer peripheral edge of the cylindrical body 46 by welding. The flange 57 and joining flange 56 provided on the lower end of the upper cauldron section 67 are fastened by bolts 58 while the ring shaped packing 48 is intervened there between. An outer diameter of the flange 57 of the night soil drying transfer pipe 54 is slightly smaller than that of the joining flange 56 of the upper cauldron section 67. Accordingly, a detachable operation between the upper cauldron section 67 and lower cauldron section 68 is made with ease via both flanges. In such a manner, the lower cauldron section 68 is closed at the longitudinal cylindrical lower end side and is opened at the upper end side while the joining flanges 56 and 57 are provided at the upper end thereof. Then the upper cauldron section 67 and lower cauldron section 68 are joined to each other via the joining flanges 56 and 57, and the ring-shaped packing 48 is intervened therebetween to fasten them by the bolts 58 so that they are integrated with each other. The drying cauldron 41 is structured as a cylindrical airtight container having a flat inner bottom which is a vertical shaft cylinder and has substantially a given fixed diameter extended in a vertical direction as a whole while it is closed at the upper and lower ends thereof.

The structures of the drying apparatuses provided in the drying cauldron 41 are described next. As shown in FIG. 7, a heater 60 for heating the cauldron is provided at the lower surface of the drying cauldron 41, namely, at the lower surface of the bottom plate 47 of the lower cauldron section 68. The heater 60 is an electric heater, and when it is energized, the interior of the drying cauldron 41 is heated to evaporate moisture of the stored night soil, thereby drying night soil. Meanwhile, an insulating member having a given thickness, not shown, is provided under the heater 60. A stirring shaft 49 serving as the stirring mechanism for stirring night soil which is sucked in the cauldron when implementing drying treatment is provided at the central position of the cauldron lid 59. The stirring shaft 49 comprises the motor 108 serving as a driving section mounted on the cauldron lid 59 and the gear box 109 in which a gear mechanism meshing with an output shaft of the motor 108 is housed. A rotating direction of the motor 108 is changed by the gear mechanism inside the gear box 109 so that the vertical stirring shaft 49 is rotated about the axis.

Stirring balls (heat accumulators) 50 stored in the drying cauldron 41:

In the drying cauldron 41, night soil is stored therein, and the drying cauldron 41 is heated by the heater 60 from the outside, thereby evaporating night soil. However, the evaporation of night soil is not smoothly implemented by merely heating the drying cauldron 41, it is necessary that night soil stored in the drying cauldron 41 is stirred to uniformly increase the temperature. Accordingly, a plurality of heat accumulators 50 for implementing stirring operation as well as assisting the heating of night soil are stored in the drying cauldron 41. The heat accumulators 50 are spherical and are made of a material formed of a metal such as stainless steel, iron, brass, or ceramics formed by sintering. Each diameter of the heat accumulators 50 is set to be in the order of one fifth to one tenth of an inner diameter of the drying cauldron 41.

A mechanism for rotating the heat accumulators (balls for drying purpose) 50 that are rotated in drying cauldron 41:

As shown in FIG. 7, the stirring shaft 49 is vertically suspended from the central position of the cauldron lid 59 in an inner space of the drying cauldron 41. That is, the stirring shaft 49 serving as a stirring means which is rotated inside the drying cauldron 41 is provided at the central portion of the drying cauldron 41 in a vertical arrangement. The stirring shaft 49 extends to a portion close to the bottom of the drying cauldron 41, and a stirring blade 110 is provided at the lower end of the stirring shaft 49. The stirring shaft 49 is rotated by the motor 108 provided over the cauldron lid 59, and the balls 50 are pushed and rotated by the stirring blade 110 provided at the lower end of the stirring shaft 49.

The motor 108 and gear box 109 are supported by a motor base 111 which is supported by the cauldron lid 59 of the drying cauldron 41. That is, a circular shaft supporting hole 112 in which the stirring shaft 49 is inserted is formed at the central position of the circular cauldron lid 59 that closes the upper end of the drying cauldron 41, and an O-ring 113 which is widened in a collar at the upper end is airtightly inserted and engaged in the shaft supporting hole 112 from above so as to be fixed thereto. A bearing case 114 for supporting the stirring shaft 49 is inserted into and fixed to the O-ring 113. The bearing case 114 is longitudinally cylindrical and comprises a barrel part 115 airtightly inserted into the drying cauldron 41 through the O-ring 113 and a flange 116 that stretches toward the upper end periphery of the barrel part 115 and is placed on the O-ring 113 to be supported thereby, wherein the flange 116 is fixed to the O-ring 113 by bolts 117. A ball bearing 118 using ceramic balls is provided on the barrel part 115 of the bearing case 114 to rotatably support the stirring shaft 49. A seal base 125 having two-stage structure is fixed to the lower end of the bearing case 114 by bolts 126, and a seal member 127 for airtightly sealing a contact portion relative to an outer peripheral surface of the stirring shaft 49 is provided on the seal base 125. As a result, a shaft supporting part of the stirring shaft 49 is kept airtight.

The motor 108 is a horizontal shaft type, and a shaft of the motor 108, not shown, is connected to the upper end of the stirring shaft 49 via a gear inside the gear box 109. Accordingly, the upper end of the stirring shaft 49 can be rotated about a vertical axis via the gear. The gear box 109 is supported on the motor base 111. The stirring shaft 49 is divided into an upper stirring shaft 128 and a lower stirring shaft 129, and the lower end of the upper stirring shaft 128 and the upper end of the lower stirring shaft 129 are connected to each other by couplings 130 and 131 to be integrally rotated. The stirring blade 110 is connected to the lower end of the lower stirring shaft 129, and it is rotated integrally with the lower stirring shaft 129 at the inner bottom portion of the drying cauldron 41, thereby rotating the heat accumulators 50.

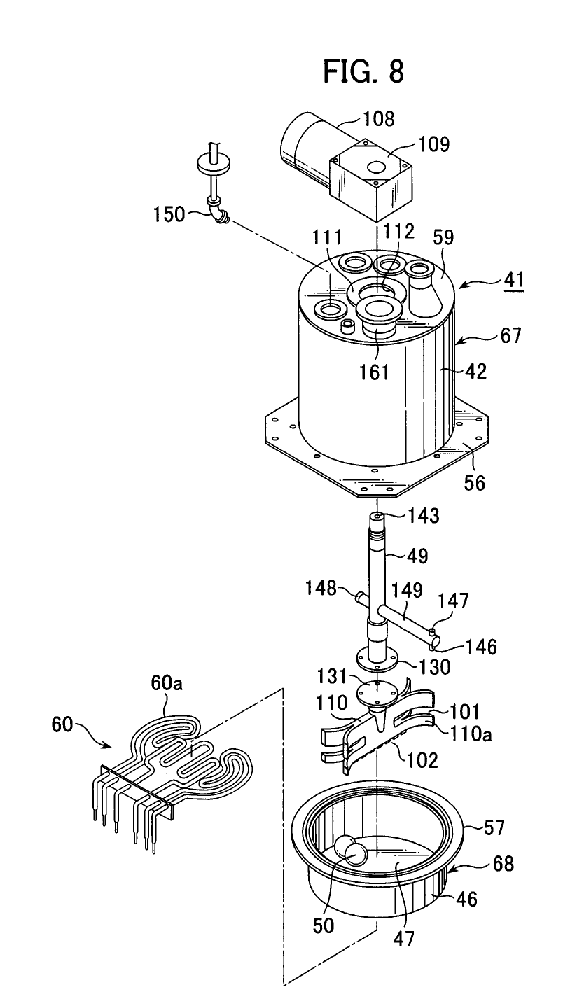

FIG. 8 shows the structures of the components of the mechanism for rotating the balls 50 in an exploded state. As shown in FIG. 8, the heater 60 for heating the drying cauldron 41 under the lower cauldron section 68 in a state where the upper cauldron section 67, lower cauldron section 68, and apparatuses provided therein are vertically separated. The heater 60 is structured that heating wires 60a are combined to each other in a width direction so as to substantially heat the entire area of the bottom plate 47 of the lower cauldron section 68. The stirring blade 110 disposed between the upper cauldron section 67 and lower cauldron section 68 are formed of two pieces of the blades that are bent at a right angle with the axis of the stirring shaft 49 so as to rotate the heat accumulators 50 disposed inside the lower cauldron section 68. Respective blade parts 110a of the stirring blade 110 have a gap 101 at the middle portion in the vertical direction, irregular parts 102 are formed vertically at the lower edges of the blade parts 10a, whereby the stirring blade 110 is rotated while partially coming into contact with the upper surface of the lower cauldron section 68, so that carbide formed after moisture of night soil is evaporated by heating is mixed so to be crushed into powder.

Washing mechanism in the drying cauldron 41:

FIG. 7 and FIG. 8 show the structure of a washing mechanism 141 as a cauldron interior washing mechanism for washing the interior of the drying cauldron 41 after night soil inside the drying cauldron 41 is dried. That is, water is jetted toward the interior of the drying cauldron 41 upon completion of drying treatment of night soil, thereby washing the interior of the drying cauldron 41 so as to remove the adherents onto the cauldron.

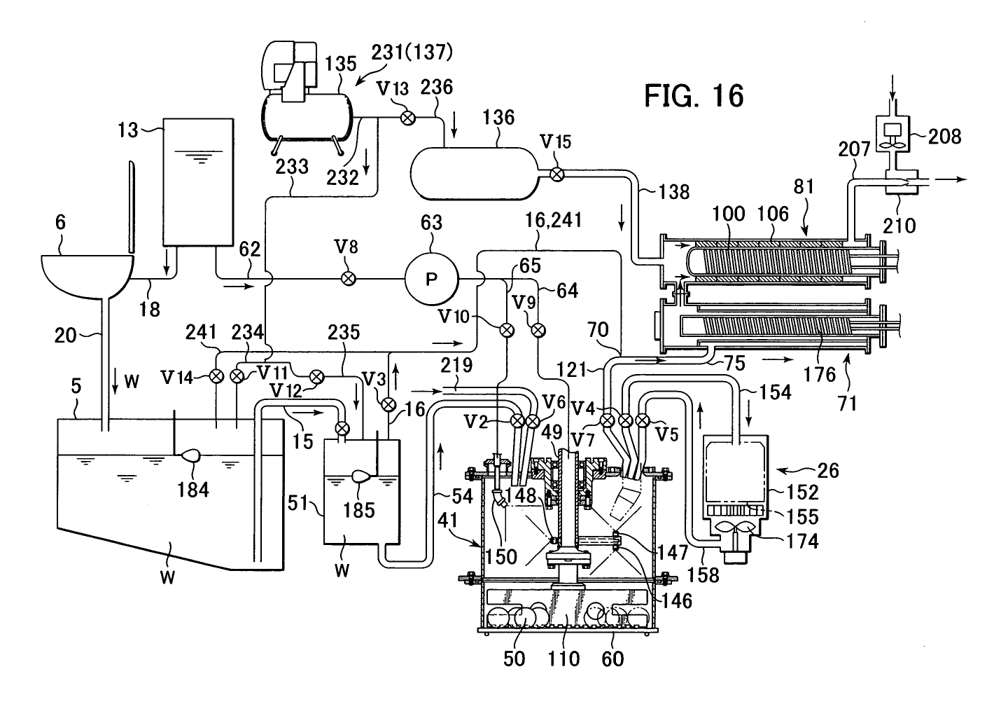

First, as shown in FIG. 7, the stirring shaft 49 which is rotated in the drying cauldron 41 and serves as the stirring means is suspended from the cauldron lid 59 and housed vertically at the central position of the drying cauldron 41. The upper stirring shaft 128 of the stirring shaft 49 is hollow and tubular so as to introduce washing water therein. That is, the upper end side of the hollow upper stirring shaft 128 is opened and has a water introduction port 142 for receiving washing water from above while the lower end of the upper stirring shaft 128 is closed by the couplings 130 and 131 serving as a connection portion relative to the lower stirring shaft 129. That is, formed inside the upper stirring shaft 128 is a cavity 143 having a longitudinal hole-shaped hollow part having a bottom capable of storing washing water therein. A given quantity of washing water can be stored in the cavity 143 for a length extending from a portion adjacent to the lower end of the upper stirring shaft 128 to the upper end thereof. A rotary joint 144 for supplying washing water is connected to the upper end of the upper stirring shaft 128. The rotary joint 144 is disposed on the upper end of the gear box 109 so as to supply washing water to a water introduction port 145 provided at the upper end of the upper stirring shaft 128 which is rotated about the perpendicular shaft. The water washing pipe 18 for supplying washing water from the water tank 13 is connected to the rotary joint 144. A high pressure water generation pump 63, described later in FIG. 16 and succeeding figures, is connected to the water washing pipe 18. The high pressure water generated by the high pressure water generation pump 63 is supplied to the water washing pipe 18, and high pressure washing water is supplied to the interior of the upper stirring shaft 128 through the water washing pipe 18 via the rotary joint 144.

A plurality of washing nozzles for receiving water while communicating with the cavity 143 so as to wash the interior of the drying cauldron 41 are provided at the lower end of the upper stirring shaft 128. These washing nozzles form the washing mechanism for jetting water toward the interior of the drying cauldron 41 upon completion of drying treatment of night soil to wash the interior of the drying cauldron 41, thereby removing the adherents onto the interior of the drying cauldron 41. More in detail, these nozzles are formed of a cauldron inner bottom washing nozzle 146, a cauldron inner ceiling washing nozzle 147 and a cauldron inner peripheral wall washing nozzle 148, and structured to be a fan-shaped jet nozzles to jet high pressure water toward entire wall surfaces of the interior of the drying cauldron 41. These washing nozzles 146, 147 and 148 are positioned at portion close to the lower end of the upper stirring shaft 128, for example, positioned at the height substantially one third of the height extending from the bottom portion to the ceiling portion inside the drying cauldron 41 at the lower portion of the drying cauldron 41. More in detail, an oblong tubular washing nozzle coupling 149 is horizontally fixed to the portion close to the lower end of the upper stirring shaft 128, and a hole 149a of the nozzle coupling 149 communicates with the cavity 143 of the upper stirring shaft 128. The length of nozzle coupling 149 is about one third of the inner diameter of the drying cauldron 41. One end of the nozzle coupling 149 along the lengthwise direction is fixed to the upper stirring shaft 128 to be integrally rotated therewith. The cauldron inner bottom washing nozzle 146 and cauldron inner ceiling washing nozzle 147 are provided at the long protruded end side of the nozzle coupling 149. The cauldron inner bottom washing nozzle 146 is provided at the lower surface of the nozzle coupling 149 and has a jetting port directed downward and structured as a fan-shaped nozzle through which washing water is spread wide in the jetting direction. Meanwhile, the cauldron inner ceiling washing nozzle 147 is provided at the upper surface of the nozzle coupling 149 and has a jetting port directed upward, and is structured as a fan-shaped nozzle through which washing water is spread wide in the jetting direction. To the contrary, the cauldron inner peripheral wall washing nozzle 148 is provided at the end opposite to the long protruded end of the nozzle coupling 149, namely, at the position close to an outer peripheral surface of the upper stirring shaft 128. The cauldron inner peripheral wall washing nozzle 148 is provided at the end of the nozzle coupling 149 and has a jetting port directed laterally and is structured as a fan-shaped nozzle through which washing water is spread wide in the jetting direction.

Accordingly, when the stirring shaft 49 is rotated in the drying cauldron 41, and high pressure water generated by the high pressure water generation pump is supplied to the water washing pipe 18, and also the high pressure washing water is supplied from the water washing pipe 18 to the upper stirring shaft 128 via the rotary joint 144, high pressure water is jetted laterally in a fan-shape from the cauldron inner bottom washing nozzle 146, cauldron inner ceiling washing nozzle 147 and cauldron inner peripheral wall washing nozzle 148 so that washing water strikes against all the ceiling, bottom surface and side surface of the drying cauldron 41 while the stirring shaft 49 is rotated, thereby washing the foreign objects such as carbide and so forth remaining in the drying cauldron 41 upon completion of drying treatment of night soil so as to be discharged in the bottom of the cauldron. As a result, the adherents such as carbide and so forth remaining in the cauldron can be washed out to be removed.

A stirring blade washing nozzle 150 is provided inside the drying cauldron 41 separately from the washing nozzles. The stirring blade washing nozzle 150 jets high pressure water from above the interior of the drying cauldron 41 toward the inner bottom side, thereby washing individually the stirring blade 110 and the surroundings thereof. For example, after completing the washing by the cauldron inner bottom washing nozzle 146, cauldron inner ceiling washing nozzle 147 and cauldron inner peripheral wall washing nozzle 148, there is a possibility that the foreign objects such as carbide and so forth remain in the stirring blade 110 disposed on the bottom of the drying cauldron 41. Accordingly, high pressure water is further jetted from the upper position of the interior of the drying cauldron 41 to enhance an jetting effect. The stirring blade washing nozzle 150 is disposed at the peripheral side of the cauldron lid 59. A washing nozzle insertion hole (e.g. circular hole) 218 through which the stirring blade washing nozzle 150 is inserted from the upper portion is bored in the cauldron lid 59, and a supporting cylinder 161 and a lid 162 are provided at the position surrounding this hole at the periphery of the washing nozzle insertion hole 218. The longitudinal tubular nozzle coupling 149 is airtightly inserted into the lid 162, and the stirring blade washing nozzle 150 is provided in a suspended state under the nozzle coupling 149. The stirring blade washing nozzle 150 is structured that a fan-shaped nozzle is provided at the tip end of an elbow-shaped pipe which is bent toward the cauldron bottom center side, and it can jet high pressure water toward the periphery from the cauldron bottom center. Although washing water from the water tank 13 is supplied to the stirring blade washing nozzle 150 by the high pressure water generation pump, high pressure water from the high pressure water generation pump flows at piping which is separated from a water supply system comprising the cauldron inner bottom washing nozzle 146, cauldron inner ceiling washing nozzle 147 and cauldron inner peripheral wall washing nozzle 148 by washing water switching valves, described later with reference to FIG. 16 and so forth. As mentioned above, the washing piping is divided into plural numbers so as to individually implement washing by switching the valves so that the washing is implemented while high water pressure is maintained.

Outline of structures of the preheating section 71, catalyst section 81 and a catalyst cleaning mechanism:

The night soil drying treatment apparatus according to the invention, as mentioned above, is provided with the preheating section 71 for heating discharge air at a constant temperature for efficiently sucking the elements of odor of discharge air from the drying cauldron 41, and catalyst section 81 for subjecting the elements of odor to the oxidation and reduction by causing the preheated discharge air to contact the catalysts and a wind pressure cleaning mechanism for blowing off the adherents onto the surfaces of catalysts by a wind pressure while supplying compressed air to the catalyst mechanism upon completion of drying treatment.

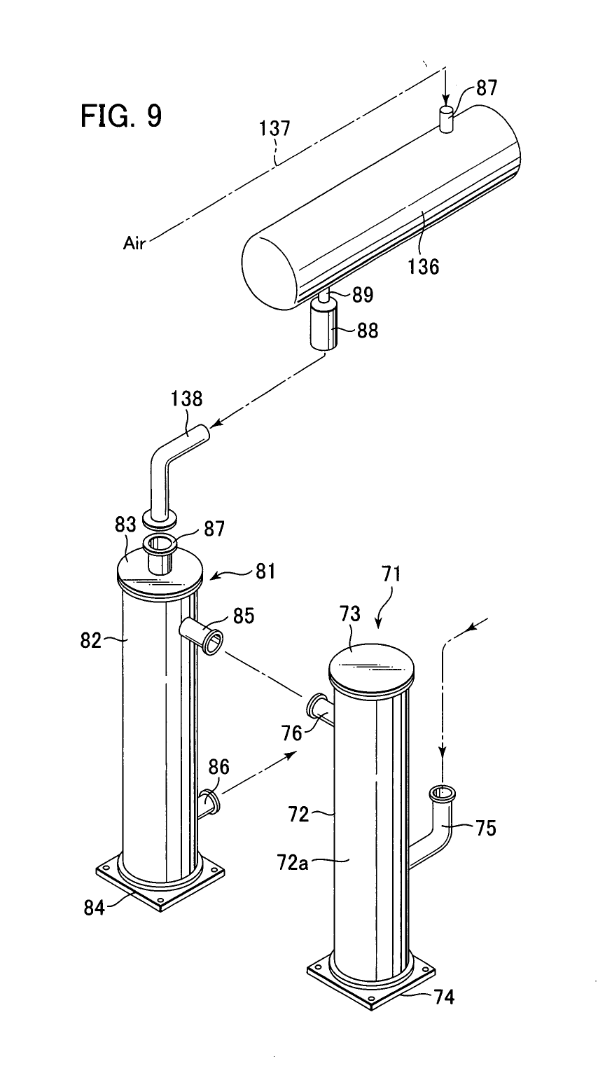

FIG. 9 is a perspective view schematically showing the preheating section 71, catalyst section 81 and constituent elements of the catalyst cleaning mechanism in an exploded manner. The preheating section 71 is illustrated at the bottom right in FIG. 9. The preheating section 71 preheats discharge air containing an odor, and as shown in FIG. 4 and FIG. 5, it has a cylindrical outer cylinder 72 which is disposed to stand upright at the left side in the housing box 12 when viewed from the front portion of the housing box 12. The outer cylinder 72 is structured that the upper end and lower end of the cylindrical body 72a are sealed by an upper lid 73 and a lower lid 74 to form an inner space while a discharge air introduction pipe 75 for introducing discharge air is provided at the position slightly lower than the central position of the outer cylinder 72 in the vertical direction. A discharge air path for causing the discharge air to flow along an axial direction and an electric heater for heating discharge air and so forth are provided inside the outer cylinder 72. Further, a discharge air feeding pipe serving as a communication pipe for feeding the heated discharge air toward the catalyst section 81 is provided at the position different from the position where the outer cylinder 72 is provided. The discharge air feed pipe 76 is provided to be positioned slightly upper side of the outer cylinder 72 in the vertical direction at the position where it is turned substantially at 90 degrees in the circumferential direction of the outer cylinder 72 relative to the discharge air introduction pipe 75. The efficiency of the layout of the respective apparatuses is enhanced in a limited space inside the housing box 12 by differentiated the connection direction of piping. The lower lid 74 for closing the lower end of the outer cylinder 72 is structured as a square base plate so as to be fixed to the bottom of the housing box 12.

The catalyst section 81 is illustrated at the bottom left of FIG. 9. The catalyst section 81 can be housed in the same housing integrally with the preheating section 71. The catalyst section 81 has a cylindrical outer cylinder 82 which is disposed to stand at the left innermost side in the housing box 12 as viewed from the front of the doors attached to the housing box 12, as shown in FIG. 4 and FIG. 5. The outer cylinder 82 is structured that the upper end and lower ends of the cylindrical body is sealed by an upper lid 83 and a lower lid 84 to form an inner space while a high temperature discharge air introduction pipe 85 for introducing high temperature discharge air after preheated is provided in the outer cylinder 82 at the position corresponding to the discharge air feed pipe 76. The outer discharge air pipe 86 is provided to be positioned in height slightly lower than the center of the outer cylinder 82 in the vertical direction at the position where it is turned substantially at 90 degrees in the circumferential direction of the outer cylinder 82 relative to the high temperature discharge air introduction pipe 85. Further, the air introduction pipe 87 for introducing compressed air when catalysts are cleaned is provided at the center position of upper lid 83 of the outer cylinder 82. Meanwhile, the lower lid 84 for closing the lower end of the outer cylinder 82 is structured, for example, as a square base plate so as to be fixed to the bottom of the housing box 12.

An air tank 136 for forming the catalyst cleaning section is illustrated at the upper portion of FIG. 9. The air tank 136 is formed of, as shown in FIG. 3 and FIG. 4, oblong cylindrical tank provided at the upper innermost side of the housing box 12 and has a compressed air introduction pipe 87 connected to a compressed air pipe 137 for introducing compressed air (Air) from the compressor 135. An air jet pipe 89 having an air operation valve 88 is provided at the other end side of the air tank 136 wherein the tip end of the air jet pipe 89 is connected to the compressed air introduction pipe 87 of the catalyst section 81. When the catalysts are washed, compressed air contained in the air tank 136 is jetted from the air jet pipe 89 to the catalysts inside the catalyst section 81 so that adherents onto the surfaces of catalysts are blown off by a wind pressure, thereby implementing catalyst cleaning operation.

Structure of the preheating section 71:

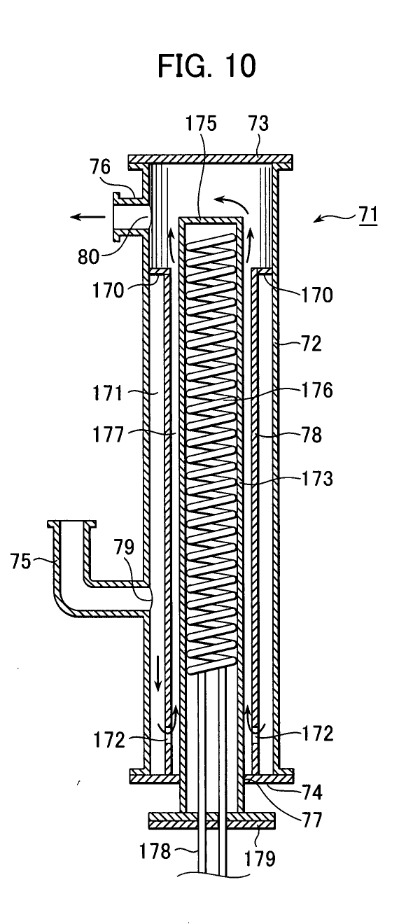

The structure of the preheating section 71 is described now in detail with reference to FIG. 10 to FIG. 12. FIG. 10 is a longitudinal sectional view wherein the outer cylinder 72 of the preheating section 71 is cut at the central axis (the discharge air feed pipe 76 provided on the outer cylinder 72 of the preheating section 71 is indicated at the same sectional position as the discharge air introduction pipe 75 which is turned right at 90 degrees from the state shown in FIG. 9). As shown in FIG. 10, the preheating section 71 has the cylindrical outer cylinder 72 which is disposed to stand upright. The upper lid 73 is provided on the upper end of the outer cylinder 72, while the lower lid 74 is provided at the lower end thereof. The upper lid 73 is a discoid airtight lid, and it completely closes the upper end opening of the outer cylinder 72. Further, the lower lid 74 is a square plate, as shown in FIG. 9 and it is structured as a base plate having a circular through hole 77 bored at the center thereof. The through hole 77 of the lower lid 74 is kept airtight by mounting an intermediate cylinder 78 thereon, described later. A suction hole 79 is penetrated to be opened at the peripheral wall of the outer cylinder 72 at the position slightly lower than the central portion in the vertical direction, and the discharge air introduction pipe 75 for sucking discharge air containing an odor to be discharged from the drying cauldron 41 and so forth is provided in the suction hole 79. An discharge air hole 80 is penetrated to be opened at the portion close to the upper end of the peripheral wall of the outer cylinder 72 in a direction, e.g. to be turned at 90 degrees relative to the discharge air introduction pipe 75 (however it is shown on the same flat surface in FIG. 10), and the discharge air feed pipe 76 is provided on the discharge air hole 80 for feeding discharge air upon completion of heating treatment. The intermediate cylinder 78 having a diameter which is about half the outer cylinder 72 and a length which is shorter than the outer cylinder 72 is provided inside the outer cylinder 72 coaxially with the outer cylinder 72. The lower end of the intermediate cylinder 78 comes into contact with the inner surface (upper surface) of the lower lid 74 and supported thereby, and the upper end of the intermediate cylinder 78 is positioned under the discharge air feed pipe 76. A ring-shaped collar 170 is integrally protruded toward an outer peripheral side from the upper end of the intermediate cylinder 78 and it comes in contact with the inner surface of the outer cylinder 72. By this, a flow path is formed inside the outer cylinder 72 partitioned by the intermediate cylinder 78 at the upper end side along the direction of the diameter, and a vertically long closed first outer cylindrical space 171 (hereinafter referred to outer cylindrical space) is provided between an inner peripheral surface of the outer cylinder 72 and an outer peripheral surface of the 78. The discharge air introduction pipe 75 communicates with the outer cylindrical space 171 via the hole 79 of the outer cylinder 72 at the position about one third of the length of the outer cylindrical space 171 from the bottom end thereof in the axial direction. Further, the peripheral wall of the intermediate cylinder 78 is penetrated to be opened at the position displaced from the axial direction of the discharge air introduction pipe 75, for example, at the position under the discharge air introduction pipe 75 to form a small diameter discharge air path hole 172. The discharge air path hole 172 is provided by plural numbers with a spaced interval in the circumferential direction of the intermediate cylinder 78. With such a structure, the discharge air containing order to be introduced from the drying cauldron 41 side first enters the outer cylindrical space 171 via the discharge air introduction pipe 75 provided in the outer cylinder 72, and moves while flows axially inside the outer cylindrical space 171 for a given length, then flows inwardly from the discharge air path hole 172 provided at the lower portion the intermediate cylinder 78.

Next, the lower lid 74 for closing the lower end of the outer cylinder 72 is opened at the center thereof to form the circular through hole 77. A heating cylinder 173 having a diameter smaller than that of the intermediate cylinder 78 is inserted into the inner side of the intermediate cylinder 78 via the through hole 77. The upper end of the heating cylinder 173 is closed by a flat lid plate 175 and the cauldron lid 59 is set at a position lower than the central position of the suction hole 79 of the discharge air introduction pipe 75 of the outer cylinder 72. A preheating heater 176 such as an electric heater is integrally built in the interior of the heating cylinder 173, and when the preheating heater 176 is heated, heating operation relative to the outer peripheral side of the heating cylinder 173 is implemented. By this, a second cylindrical space (hereinafter referred to as inner cylindrical space) 177 is formed between the intermediate cylinder 78 and heating cylinder 173. In such a manner, the inner cylindrical space 177 is formed between the heating cylinder 173 and the intermediate cylinder 78, so that discharge air flown inwardly from the outer cylindrical space 171 via the discharge air path hole 172 enters the inner cylindrical space 177, as shown in an arrow in FIG. 10, and flows upward axially along the outer peripheral surface of the heating cylinder 173. The discharge air is heated during the upward flowing. Heated discharge air is fed from the discharge air feed pipe 76 provided at the upper end side of the outer cylinder 72 to the outside adjacent to the catalyst section 81 side. Meanwhile, the inner cylindrical space 177 forms a primary heating area by the heating cylinder 173 while the preheating heater 176 serves as a heat source, the outer cylindrical space 171 forms a secondary heating area via the inner cylindrical space 177 while the preheating heater 176 serves as a heat source. That is, discharge air, that flows reciprocally in the preheating section 71 while meandering inwardly from the outside along the axial direction of the preheating section 71, is first heated by the secondary heat source of the preheating heater 176 and heating cylinder 173 while flowing in the outer cylindrical space 171, then heated by the primary heat source of the preheating heater 176 and heating cylinder 173 while flowing in the inner cylindrical space 177, whereby discharge air is efficiently heated by the two-stage heating and fed to the catalyst section 81 as heated discharge air.

An outer diameter of the heating cylinder 173 is substantially the same as the diameter of the through hole 77 formed at the central portion of the lower lid 74, and the outer surface of the heating cylinder 173 is inserted into the through hole 77 of the lower lid 74 in a state of close contact with the through hole 77. Further, the lower end side portion of the heating cylinder 173 protrudes under the outer cylinder 72. The lower end side at the portion protruded downward from the outer cylinder of the heating cylinder 173 is opened, and the preheating heater 176 is inserted into substantially the entire area of the interior of the heating cylinder 173. A terminal wires 178 of the preheating heater 176 extend under the heating cylinder 173, and they are held by a holding plate 179 disposed under the outer cylinder 72. The holding plate 179 serves as a lid plate for closing the interior of the heating cylinder 173.

Constituent members of the preheating section 71:

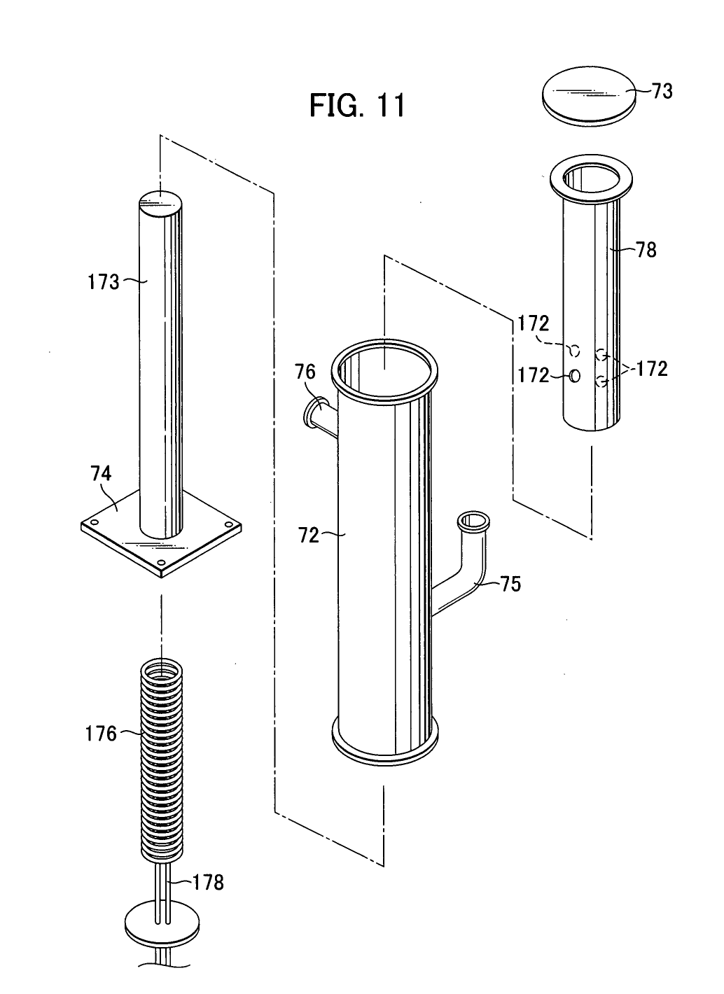

FIG. 11 is a perspective view showing an exploded structure of the constituent members of the preheating section 71. At the center of FIG. 11, the cylindrical outer cylinder 72 disposed while standing upright is shown. Both the upper and lower ends of the outer cylinder 72 are opened, and the discharge air introduction pipe 75 protrudes from the peripheral wall of the outer cylinder 72 at the lower end side, while the discharge air feed pipe 76 protrudes from the peripheral wall of the outer cylinder 72 at the upper end side and arranged to be turned 90 degrees relative to the discharge air introduction pipe 75. There is disclosed at the top right of FIG. 11, the circular upper lid 73 for closing the upper end opening of the outer cylinder 72 and the intermediate cylinder 78 to be inserted into the outer cylinder 72. A plurality of discharge air path holes 172 are bored on the intermediate cylinder 78 at the lower portion in the circumferential direction with a spaced interval. At the top left of FIG. 11, the heating cylinder 173 and lower lid 74 are illustrated, while at the bottom left of FIG. 11, the preheating heater 176 and holding plate 179 of the preheating heater 176 are illustrated. When these constituent members are assembled, the preheating section 71 shown in FIG. 10 is structured.

Structure of the catalyst section 81:

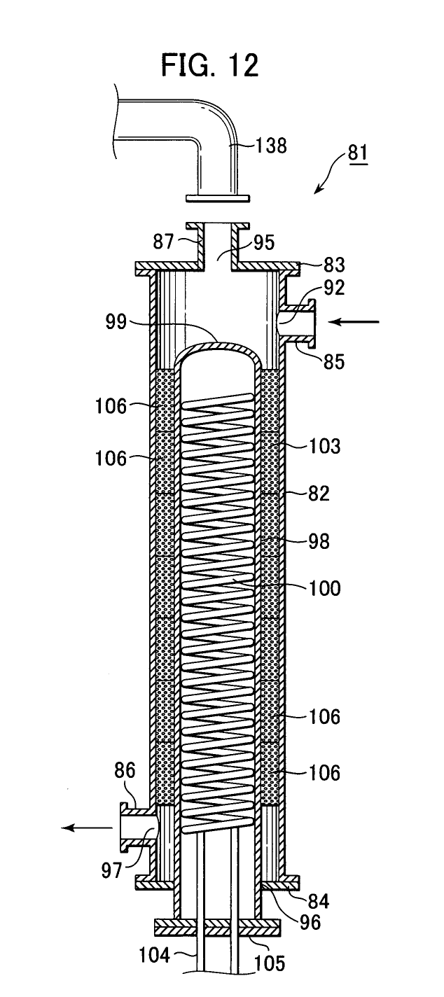

FIG. 12 is a longitudinal sectional view showing the structure of the catalyst section 81, and it is a longitudinal sectional view by cutting the outer cylinder 82 of the catalyst section 81 along the central axis portion. Meanwhile, the outer discharge air pipe 86 provided on the outer cylinder 82 of the catalyst section 81 is indicated at the same sectional position as the high temperature discharge air introduction pipe 85 by turning it 90 degrees at the state shown in FIG. 9, thereby smoothing the explanation.

The catalyst section 81 has the outer cylinder 82 having the same length and diameter as those of the high temperature discharge air introduction pipe 85. The outer cylinder 82 is a vertical axis type and structured by a cylinder made of corrosion resistant metal and is opened at the upper and lower ends. The outer cylinder 82 has the upper lid 83 at the upper end and the lower lid 84 at the lower end. The upper lid 83 is circular and is opened at the center to form a cleaning air introduction hole 95. The cleaning air introduction pipe 87 is provided on the cleaning air introduction hole 95 and it is connected to the discharge pipe 138 at the air tank 136 side. Further, the outer cylinder 82 is penetrated to be opened at the portion close to the upper end of the peripheral wall thereof to form a high temperature discharge air introduction hole 92 for introducing high temperature discharge air heated at the preheating section. The high temperature discharge air introduction pipe 85 is provided in the high temperature discharge air introduction hole 92. The lower lid 84 is structured as a base plate by boring a square plate to form a circular hole 96 at the central portion, and the circular hole 96 of the lower lid 84 is kept airtight by mounting a heater thereon, described later. The peripheral wall of the outer cylinder 82 is opened to form a discharge air hole 97 which is directed laterally and positioned at the lower end side of the outer cylinder 82 and the outer discharge air pipe 86 is provided in the discharge air hole 97. The outer periphery side of the outer cylinder 82 is covered with an insulating material, not shown.

A heating cylinder 98 having a diameter of about one half of that of the outer cylinder 82 is inserted into the circular hole 96 formed at the central portion of the lower lid 84 which closes the lower end of the outer cylinder 82. The heating cylinder 98 is formed of a corrosion resistant metal. When the heating cylinder 98 is inserted into the circular hole 96 of the lower lid 84, the circular hole 96 is kept airtight. The heating cylinder 98 is formed of an integrated member and the upper end thereof is formed of a dome-shape. The top portion of this dome is set to be substantially the same height as central position of the hole of the high temperature discharge air introduction pipe 85 of the outer cylinder 82. A catalyst heater 100 such as an electric heater is inserted into and assembled with the interior of the heating cylinder 98, wherein the heating cylinder 98 is heated from the inner side by the catalyst heater 100, so that the heating operation toward the outside of the heating cylinder 98 is implemented. By the insertion of the heating cylinder 98, a cylindrical space 103 is formed between the outer cylinder 82 and heating cylinder 98. The lower end side portion of the heating cylinder 98 protrudes under the outer cylinder 82. The lower end side of the portion protruded under the outer cylinder 82 of the heating cylinder 98 is opened, and the catalyst heater 100 is inserted into substantially entire interior area of the heating cylinder 98. Terminal wires of the catalyst heater 100 extend under the heating cylinder 98, and they are held by a holding plate 105 disposed under the outer cylinder 82. The holding plate 105 serves as a lid plate for closing the interior of the heating cylinder 98.

The catalysts 106 for subjecting the elements of odor to oxidation and reduction are stored in the cylindrical space 103 formed by the outer cylinder 82 and heating cylinder 98. The catalysts 106 are each formed of hollow metallic catalysts which are disposed in the periphery of the heating cylinder 98 along its axial direction, and they are disposed and fixed between the position of the high temperature discharge air introduction hole 92 of the high temperature discharge air introduction pipe 85 in the cylindrical space 103 and the position of the discharge air hole 97 of the outer discharge air pipe 86, respectively in the axial direction. The catalysts 106 are formed by connecting a plurality of (e.g. 7 pieces of) ring-shaped catalyst elements and detachably fixed to the periphery of the heating cylinder 98 by a holding member, not shown.

With the structure as mentioned above, when the thickness of each catalyst 106 in the circumferential direction is thinned and the metallic catalysts are used, heat of the catalyst heater 100 disposed inside the heating cylinder 98 is uniformly transferred circumferentially, and the temperature of each catalyst element is less varied, thereby obtaining uniform heating. Further, since the metallic catalysts are used as the catalysts 106, heat conduction is enhanced, the catalysts 106 can be heated in a short time up to a catalyst activation temperature with low heat capacity. Further, since the edge of each catalyst 106 is thin and an area where gas passes is wide, a pressure loss is small and dust and so forth is hardly clogged. Further, since each catalyst 106 has a hollow structure and the catalyst heater 100 is disposed at the central portion of the catalyst section 81, the catalyst section 81 can be small sized.

According to the present embodiment, the drying cauldron 41 is heated to evaporate night soil inside the drying cauldron 41, and the discharge air mechanism is provided for sucking air and moisture evaporated in the drying cauldron 41 while the catalyst section 81 is intervened between the drying cauldron 41 and discharge air mechanism for subjecting the elements of odor to oxidation and deduction, and the odor pipe is connected between an inflow side of the catalyst section 81 and the upper space of the night soil tank 5 for causing air to flow, whereby air and vapor are discharged outside by the discharge air mechanism during vaporizing drying treatment of night soil in the drying cauldron 41, and the elements of odor is subjected to oxidation and reduction by the catalyst section 81 in the midway of discharge of air and vapor, and simultaneously air in the upper spaces of the night soil tank 5, piping and drying cauldron 41 and so forth is sucked by the odor pipe to cause to pass through the catalyst section 81, thereby causing the odor to be odorless in the space of the night soil tank 5 and so forth.

Constituent members of the catalyst section 81:

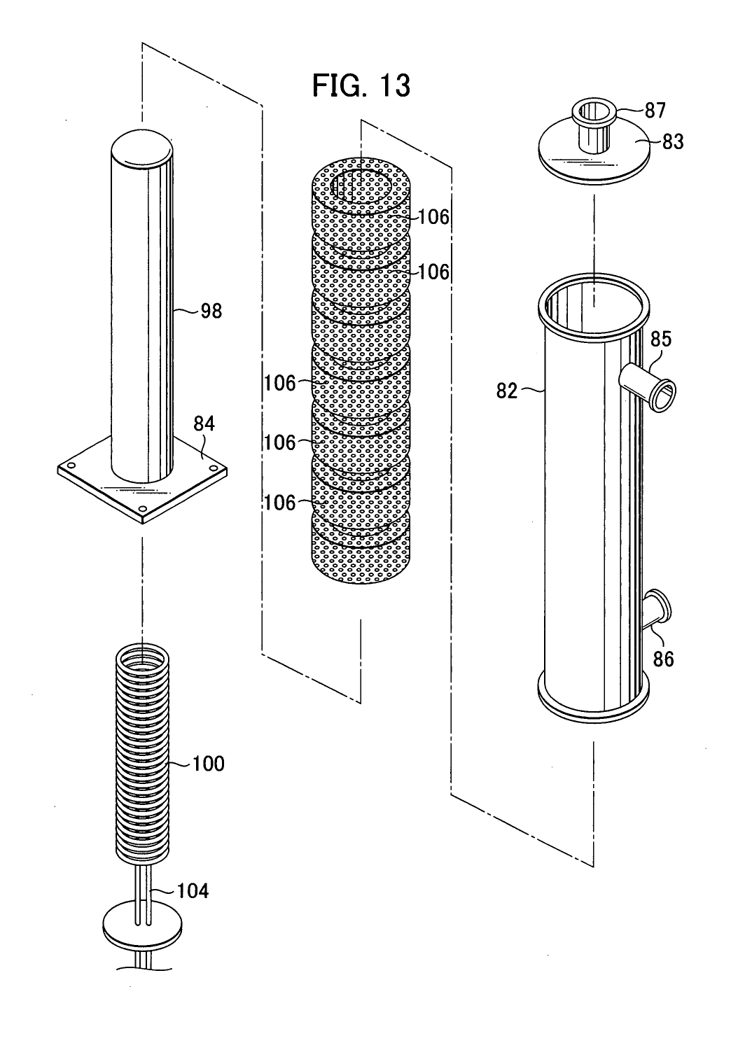

FIG. 13 is a perspective view showing an exploded structure of the constituent members of the catalyst section 81. At the right side of FIG. 13, there are illustrated a cylinder 82 disposed while standing upright and a circular upper lid 83 for closing the upper opening of the outer cylinder 82. The upper and lower ends of the outer cylinder 82 are opened, and the high temperature discharge air introduction pipe 85 is protruded from the peripheral wall of the outer cylinder 82 at the upper end side, and the other discharge air pipe 86 is protruded from the peripheral wall of the outer cylinder 82 at the lower end side and disposed at the position turned 90 degrees relative to the high temperature discharge air introduction pipe 85. A cleaning air introduction pipe 87 for introducing compress air for cleaning the catalysts is provided on the upper lid 83. At the center of FIG. 13, there is illustrated a structure of the catalysts 106 comprised of seven hollow metallic catalysts arranged in a cylinder. Further, at the top left of FIG. 13, the heating cylinder 98 and lower lid 84 are illustrated while at the bottom left, the catalyst heater 100 is illustrated. When these constituent elements are assembled, the catalyst section 81 shown in FIG. 12 is structured.

Structure of the dust collection section 26:

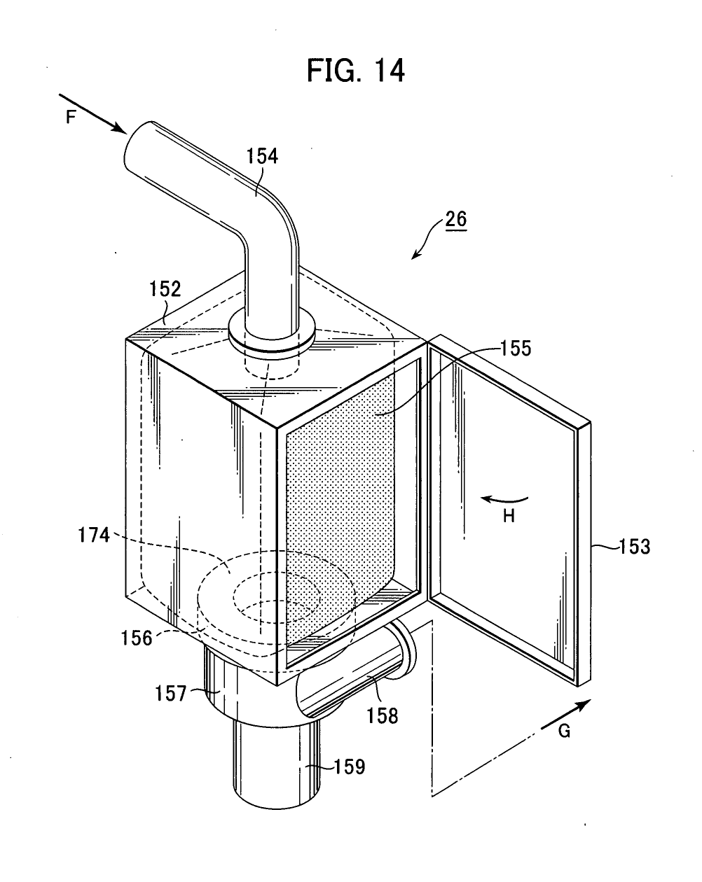

Then, the structure of the dust collection section 26 is described with reference to FIG. 14. The dust collection section 26 has a structure similar to a generally used electric cleaner, and it can capture dust from air and cause clean air alone to flow outside.

An outer shell of the dust collection section 26 comprises a collection box 152 and a side lid plate 153, and a cubic structure which is hollow inside is formed by the collection box 152 and side lid plate 153. The collection box 152 is formed by bending a thin steel sheet and one side surface (front right side in FIG. 14) is opened, wherein one side surface is largely opened but the upper and lower surfaces and other peripheral three surfaces are closed by a flat sheet in a box-shape. The side lid plate 153 is formed by bending the thin steel sheet and a peripheral edge thereof is bent shallow to form a shallow lip-shape and has substantially the same shape as the opened side surface of the collection box 152. The side lid plate 153 is connected to the right side of the side opening of the collection box 152 at the right side by a pin so as to be freely turned, and it is connected to the collection box 152 so as to be turned right and left about the pin. FIG. 14 shows a state where the side lid plate 153 is pulled from the collection box 152 right forward so that the side opening of the collection box 152 is opened. When the side lid plate 153 is turned in H direction in FIG. 14, the side lid plate 153 can be brought into intimate contact with the side surface of the collection box 152, so that the outer shell which is kept airtight from the outside can be assembled by fitting the collection box 152 to the side lid plate 153. The side lid plate 153 can be turned about the pin, thereby causing the side surface of the collection box 152 to be exposed outside at the time of maintaining and inspecting the dust collection section 26.

The upper surface of the collection box 152 is flat and square, and an introduction pipe 154 communicating with the interior of the collection box 152 is fixed to the upper central surface of the collection box 152 at the center thereof. A dust bag 155 formed of a breathing material such as a cloth or paper which is sewn in cubic shape is housed in an inner space of the collection box 152 at the upper portion thereof. The dust bag 155 has a shape which is closed from the outside, and the upper portion thereof is connected to the lower end of the introduction pipe 154, thereby causing the introduction pipe 154 and the inner space of the dust bag 155 to communicate with each other. The dust bag 155 is formed of a coarse material such as a cloth or paper and so forth, thereby causing air to flow through a film surface thereof, but it has a function to capture dust at the surface thereof. Accordingly, air introduced from the introduction pipe 154 into the interior of the dust bag 155 passes through the film surface of the dust bag 155, and dust mixed with air is captured by the film surface of the dust bag 155, so that cleaned air alone flows out in the inner space of the collection box 152.

Further, a filter 156 formed of a fine wire gauze is provided on a bottom surface of the interior of the collection box 152, and it can collect rubbish and dust having relatively large size. A fan 174 is built in the lower surface of the collection box 152 at the center thereof, and a suction device 157 for sucking air inside the collection box 152 toward outside is fixed to the lower surface of the collection box 152. A discharge pipe 158 for discharging air sucked from the collection box 152 toward the direction of G in FIG. 14 is connected to the outer surface of the suction device 157, and a motor 159 for driving the fan 174 inside the suction device 157 is fixed to the lower surface of the suction device 157.

Connecting state of piping to the drying cauldron 41 in the night soil treatment apparatus 11:

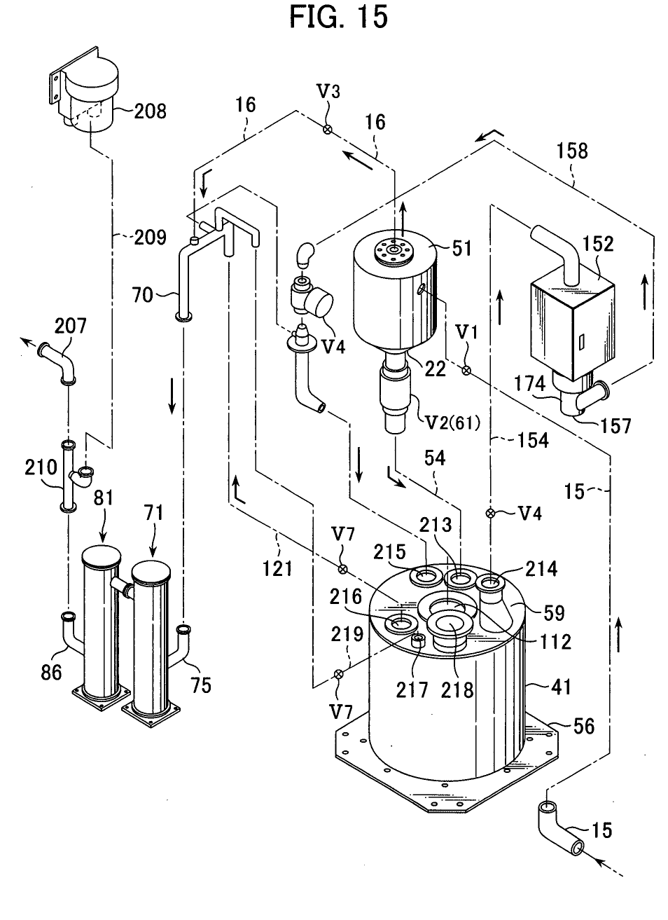

Described next with reference to FIG. 15 is a state of connection of piping for causing night soil and air between the drying cauldron 41 and the apparatuses connecting thereto among the apparatuses constituting the night soil treatment apparatus 11 of the temporary toilet 1 to which the present embodiment is applied. The arrangements of the apparatuses in the vertical and lateral directions in FIG. 15 are substantially the same as those of the apparatuses shown in FIG. 4.

Described first is holes for use in connection of piping provided in the drying cauldron 41. As shown in FIG. 15, a plurality of holes are formed circumferentially on the circular cauldron lid 59 of the drying cauldron 41 with a spaced interval and disposed about a stirring shaft insertion hole 112 (shaft supporting hole in FIG. 7). These plurality of holes are six in total. A night soil introduction hole 212 for introducing night soil to be fed from the quantitative tank 51 into the drying cauldron 41 is formed substantially over the stirring shaft insertion hole 112 provided at the center of the cauldron lid 59. A dust collection suction hole 214 and an air return hole 215 for sucking air inside the drying cauldron 41 outside and circulating it so as to collect dust such as carbide powder and so forth remaining in the drying cauldron 41, and an odor discharge hole 216 for discharging the odor from the drying cauldron 41 provided adjacent to the air return hole 215 in a counterclockwise as viewed from the air return hole 215 are formed at both sides of the cauldron lid 59. Likewise, an open air introduction hole 217 for introducing the open air inside the drying cauldron 41 when cooling the drying cauldron 41, a washing nozzle insertion hole 218 for inserting the stirring blade washing nozzle for implementing cleaning are respectively formed in the cauldron lid 59.

Next, FIG. 15 schematically shows the structure of a piping system of the apparatuses shown in FIG. 4, such as night soil transfer piping (night soil transfer pipe) 15 for transferring night soil from the quantitative tank 51 in which night soil is temporarily stored to the drying cauldron 41, the dust collection piping (introduction pipe) 154 for sucking air inside the drying cauldron 41 from the drying cauldron 41 and introducing into the dust collection section 26 disposed outside, the air return piping (discharge pipe) 158 for returning air again, which air is cleaned after dust is collected in the dust collection section 26, to the drying cauldron 41 or a discharge air piping 207 for sucking air inside the drying cauldron 41 by the preheating section 71 or catalyst section 81 to adsorb elements of odor in air, thereby eliminating the odor and odorless air is discharged outside the night soil treatment apparatus 11.

Each piping is described in detail hereafter. The night soil suction piping (night soil transfer pipe) 15 for sucking night soil from the night soil tank (omitted in FIG. 15) provided under the toilet stool 6 shown in FIG. 4 toward quantitative tank 51 is illustrated at the right side of the FIG. 15. The tip end of night soil suction piping 15 is connected to the upper portion of the quantitative tank 51 shown in the central portion of FIG. 15. A closing valve v1 formed of an electromagnetic valve is provided in the night soil transfer piping (night soil transfer pipe) 15 wherein when the closing valve v1 is controlled by a control mechanism to be opened or closed, a given quantity of night soil which is set to implement one time drying treatment in the drying cauldron 41 is supplied to the interior of the quantitative tank 51. The night soil discharge port 22 is provided at the lower portion of the quantitative tank 51, and the night soil drying transfer pipe 54 is connected to the night soil discharge port 22. A closing valve v2 (61) is provided in the night soil drying transfer pipe 54. The tip end of the night soil drying transfer pipe 54 is inserted into the drying cauldron 41 via a night soil introduction hole 213 of the cauldron lid so that night soil to be subjected to drying treatment is supplied to the drying cauldron 41. When night soil drying treatment is implemented, the closing valve v2 is controlled by the control mechanism so that a given quantity of night soil to be subjected to drying treatment is supplied to the drying cauldron 41.

Described next is a piping system for discharging the odor from the drying cauldron 41. An end of the discharge air piping 121 is connected to the odor discharge hole 216 of the drying cauldron 41 and the other end of the discharge air piping 121 is connected to a discharge air inlet part (discharge air introduction pipe 75) of the preheating section 71 shown in the bottom left of FIG. 15. A closing valve v7 is provided in the discharge air piping 121. The preheating section 71 is provided at the pre-stage of the catalyst section 81, as mentioned above, to preheat discharge air containing elements of odor to activate it so as to efficiently implement oxidation and reduction reaction at the catalyst section 81. To that end, the preheating section 71 is heated prior to the drying treatment of night soil, and it has a function to suck air in the discharge air piping 121 by always heating air. Further, discharge air port (outer discharge air pipe 86) for discharging air to the open air side is provided in the catalyst section 81 communicating with the preheating section 71, and a fan 208 and the discharge air piping 207 which is disposed on an air jetting port of the fan 208 via a fan pipe 209 are provided in the outer discharge air pipe 86 as shown in the left side of the FIG. 15 whereby compulsory discharge air operation directing from the catalyst section 81 to the open air side is implemented by the discharge air piping 207. Accordingly, air containing the odor in the drying cauldron 41 is always sucked toward the preheating section 71 side and is discharged to the open air side.

The odor discharge piping 16 extended from the upper portion of the quantitative tank 51 for temporarily storing night soil is connected to the discharge air piping 121 for discharging the odor from the drying cauldron 41 to the preheating section 71 side by a three directional pipe coupling 70. A suction force is generated by preheating at the preheating section 71 side and a negative pressure caused by a suction operation of an ejector 210 of the catalyst section 81 against the open air side at the connection portion between the discharge air piping 121 and the odor discharge pipe 16. Accordingly, the suction operation is generated in the upper inner space of the quantitative tank 51 via the odor discharge pipe 16 so that air containing the elements of odor inside the quantitative tank 51 is sucked toward the discharge air piping 207 side and is discharged to the open air side. Meanwhile, a closing valve v3 is provided in the odor discharge pipe 16. The closing valve v3 is controlled by a control device and is normally opened so as to discharge the odor, but it is closed when night soil is transferred from the quantitative tank 51 to the drying cauldron 41. The reason is that, as shown in FIG. 16, described hereafter, compressed air from the compressor 135 is fed to a liquid surface of night soil inside the quantitative tank 51, and night soil inside the quantitative tank 51 is supplied to the drying cauldron 41 by a pressurized force of the compressed air so that the interior of the quantitative tank 51 is needed to be airtight.

Described next is a piping system of the dust collection section 26 for collecting dust such as carbide and so forth remaining in the drying cauldron 41 by sucking air inside the drying cauldron 41 outside to be circulated upon completion of drying treatment. The collection box 152 described with reference to FIG. 14 is shown in FIG. 15 at the top right thereof. The introduction pipe 154 of the collection box 152 is connected to the dust collection suction hole 214 provided on the cauldron lid 59 of the drying cauldron 41. The suction device 157 having the fan 174 built therein is provided at the lower surface central portion of the collection box 152, wherein dust and so forth inside the drying cauldron 41 is sucked in the collection box 152 by the suction operation via the introduction pipe 154. Further, the collection return piping (discharge air pipe) 121 is connected to the collection box 152 at the discharge air side, and it is connected to the air return hole 215 provided on the cauldron lid 59 of the drying cauldron 41. Air from which dust and so forth is removed by the discharge air piping 121 is returned to the drying cauldron 41. Closing valves v4 and v5 are provided in the introduction pipe 154 and discharge air pipe 58, respectively, so as to control the quantity of collected air.

Further, as shown in the bottoms of FIG. 15, an open air introduction hole 217 is opened at the cauldron lid 59 of the drying cauldron 41 for introducing the open air, and an air introduction piping (open air introduction pipe) 219 is provided in the open air introduction hole 217. The air introduction piping 219 is opened to the open air and has a closing valve v6. The closing valve v6 is always closed and is opened during a cooling step upon completion of drying treatment in the drying cauldron 41, thereby enhancing cooling by introducing the open air into the drying cauldron 41. Meanwhile, in the FIG. 15, the stirring shaft insertion hole 112 is opened at the central portion of the cauldron lid 59 of the drying cauldron 41 through which the stirring shaft 49 shown in FIG. 7 is inserted. The stirring shaft 49 inserted into the stirring shaft insertion hole 112 is driven by the motor 108, described before, and it is rotated in the drying cauldron 41 which is heated during night soil drying treatment, thereby causing the heat accumulators (balls) 50 to be moved inside the drying cauldron 41 by the stirring blade 110 provided at the lower end of the stirring shaft 49 so as to stir night soil. Further, the stirring shaft 49 has a hollow tubular structure, as mentioned above, and it introduces washing water in the cavity 143 serving as an interior hole, so that the interior of the drying cauldron 41 can be cleaned by supplying washing water to the washing water nozzle. Still further, the hole illustrated under the stirring shaft insertion hole 112 in FIG. 15 is a washing nozzle insertion hole 218 through which the stirring blade washing nozzle 150 shown in FIG. 14 is inserted when the interior of the drying cauldron 41 is cleaned upon completion of drying treatment.

Structure of the connection of piping of the entire apparatus:

FIG. 16 shows the structure of the connection of the entire piping of the entire apparatus. Here, the structure of the connection of piping is divided into (1) a structure of piping of a night soil transfer system (hereinafter referred to as night soil transfer system piping), (2) a structure of piping of a water supply system (hereinafter referred to as water supply system piping) and (3) a structure of piping of an air system (hereinafter referred to as air system piping), which are described sequentially. There is a case where a part of structures of piping is combined with another structure of piping in view of the function of each system piping.

In FIG. 16, the toilet stool 6 is shown at the left end portion of the same figure and the water tank 13 is shown at the upper right side of the toilet stool 6 while the night soil tank 5 is shown immediately under the toilet stool 6. The quantitative tank 51 is shown at the portion adjacent to the right side of the night soil tank 5. The drying cauldron 41 is shown at the lower center of FIG. 16 and positioned at the position adjacent to right side of the quantitative tank 51, and the dust collection section 26 is shown at the right side of the drying cauldron 41. The preheating section 71 and catalyst section 81 are shown over the dust collection section 26 in a state where they are overturned 90 degrees and laid on its side to be oblong from the state shown in FIG. 4, FIG. 5, and FIG. 9 to FIG. 15, and they are disposed in two stages. The compressor 135 and air tank 136 are respectively shown at the top left side and center of FIG. 16.

Structure of the night soil transfer system piping:

The night soil tank 5 is disposed under the toilet stool 6. A peripheral wall of the night soil tank 5 is structured airtight and the interior of the night soil tank 5 is kept airtight. A night soil exit of the toilet stool 6 and a night soil inlet provided on the upper portion of the night soil tank 5 are connected to each other by the night soil pipe 20. The night soil pipe 20 is provided with a valve, not shown, capable of airtightly closing the interior of the night soil pipe 20, and the interior of the night soil pipe 20 is airtightly closed after night soil is discharged in the night soil tank 5. The toilet stool 6 is a flushing toilet stool, and the water washing pipe 18 for supplying washing water from the water tank 13 is connected to the toilet stool 6. Night soil W excreted from a utilizer is stored inside the night soil tank 5 together with flushing water. A night soil sensor (e.g. float sensor) 184 for automatically detecting the quantity of night soil stored inside the night soil tank 5 is provided in the night soil tank 5. A detection signal representing the quantity of stored night soil is outputted from the night soil sensor 184 to a control section, described later. A sensor, not shown, for detecting the quantity of remaining night soil is also provided at the inner bottom portion side of the night soil tank 5, and a signal noticing the quantity of night soil remaining in the night soil tank 5 is outputted from the night soil sensor to the control section, described later.

The night soil transfer pipe 15 is provided between the night soil tank 5 and quantitative tank 51 so as to connect therebetween. The night soil suction port provided at one end of the night soil transfer pipe 15 is disposed at the inner bottom portion of the night soil tank 5 so as to suck night soil stored in the night soil tank 5. The night soil discharge port provided at the other end of the night soil transfer pipe 15 is airtightly connected to the cauldron lid for keeping the upper end of the quantitative tank 51 airtight, and it is opened downward at the interior of the quantitative tank 51. The closing valve v1 is provided in the night soil transfer pipe 15. The closing valve v1 is subjected to opening and closing control in response to an instruction signal from the control section so that it is controlled to transfer the quantity of night soil which is set for a load of night soil to be subjected to drying treatment in the drying cauldron 41. That is, if it is decided that the quantity of the night soil stored inside the night soil tank 5 at the toilet stool 6 side reaches a set quantity of night soil to be subjected to drying treatment, the closing valve v1 is opened so that the quantity of night soil as set for one load of night soil to be subjected to drying treatment in the drying cauldron 41 is transferred to the quantitative tank 51. The closing valve v1 is closed after the set quantity of night soil is stored in the quantitative tank 51. A night soil sensor 185 for automatically detecting the quantity of night soil to be transferred is provided in the quantitative tank 51 so as to control the closing valve v1. The night soil sensor 185 is a float sensor and a detection signal representing the quantity of night soil in the quantitative tank 51 is outputted from the night soil sensor 185 to the control section. A detection signal representing the quantity of transfer of night soil is outputted from the night soil sensor 185 to the control section in response to the detection signal representing the quantity of night soil to control the closing valve so that the quantity of night soil is stored in the quantitative tank 51.

Further, there is provided the night soil drying transfer pipe 54 for connecting the quantitative tank 51 and drying cauldron 41. The night soil suction port provided at one end of the night soil drying transfer pipe 54 is disposed at the inner bottom portion of the quantitative tank 51 so that it can suck the entire quantity of night soil stored in the quantitative tank 51. The night soil discharge port provided at the other end of the quantitative tank 51 is airtightly connected to the cauldron lid 59 for keeping the upper end of the drying cauldron 41 airtight, and the night soil discharge port is opened downward toward the interior of the drying cauldron 41. The closing valve v2 is provided in the night soil drying transfer pipe 54. The closing valve v2 is subjected to opening and closing control in response to an instruction signal from the control section so as to be controlled to transfer the quantity of night soil as set for one load of night soil to be subjected to drying treatment in the drying cauldron 41. That is, it is set such that the entire quantity of night soil stored in the quantitative tank 51 is transferred to the drying cauldron 41, thereafter the closing valve v2 is closed.

(2) Structure of the water supply system:

A water supply source of the water supply system is the water tank 13 from which two pipes are connected, and one pipe is the water washing pipe 18. The water washing pipe 18 supplies flushing water to the toilet stool 6 to wash the interior of the toilet stool 6, and it is connected to the toilet stool 6 as mentioned in the above column (1). The other pipe is a cauldron washing pipe 62 for supplying washing water to the interior of the drying cauldron 41 so as to wash the drying cauldron 41. A high pressure water generation pump 63 is provided in the cauldron washing pipe 62 via a closing valve v8, and the cauldron washing pipe 62 is branched into two at the downstream side of the high pressure water generation pump 63. A branch pipe 64 which is one branch of the cauldron washing pipe 62 is connected to the stirring shaft 49 so as to supply water to the washing nozzles 146, 147 and 148 of the stirring shaft 49 provided at the central position of the cauldron lid 59 of the drying cauldron 41 (more in detail, the branch pipe 64 is connected to the rotary joint 144 provided at the upper end of the stirring shaft 49, not shown in FIG. 16). A branch pipe 65 which is the other branch of the cauldron washing pipe 62 is connected to the other nozzle provided at the peripheral side of the cauldron lid 59, namely, to the stirring blade washing nozzle 150, described above. The closing valve v8 is provided at the cauldron washing pipe 62 at the upstream side of the high pressure water generation pump 63, wherein a flow path at the water tank 13 side serving as a water supply source is opened and closed by the closing valve v8. Further, closing valves v9 and v10 are provided in the branch pipes 64 and 65, respectively, branched at the downstream side of the high pressure water generation pump 63. The closing valve v9 of one branch pipe 64 connected to the stirring shaft 49 is opened when the interior surface of the drying cauldron 41 is cleaned to supply washing water to the interior of the drying cauldron 41. The closing valve v9 is closed upon completion of washing the drying cauldron 41 to stop the supply of washing water. On the other hand, the closing valve v10 of the other branch pipe 65 at the stirring blade washing nozzle 150 side provided at the different position of the drying cauldron 41 is opened upon completion of washing the interior surface of the drying cauldron 41 to wash the stirring blade 110 inside the drying cauldron 41, and it is closed upon completion of washing the stirring blade to stop the supply of washing water.

(3) Structure of the air system piping:

The air system piping is roughly divided into four types. The first air system piping comprises a compressor 135 and an air pressure system piping 231 for connecting between the compressor 135, night soil tank 5 and quantitative tank 51. The air pressure system piping 231 is piping for selectively supplying compressed air from the compressor 135 to the night soil tank 5 and the quantitative tank 51 so as to pressurize the interior of the night soil tank 5 or the interior of the quantitative tank 51 so that night soil is transferred from the night soil tank 5 to the quantitative tank 51 or from the quantitative tank 51 to the drying cauldron 41 by pressurizing the surface of night soil stored in the drying cauldron 41. The pressure system piping 231 comprises a piping 232 for cleaning air connected to the compressor 135 for causing compressed air discharged from the compressor 135 to flow, a compressed air branch piping 233 for transferring night soil comprised of one of branch pipes which are branched from the piping 232 for cleaning air, and a night soil tank inner pressure application piping 234 which is connected to the night soil tank 5 and quantitative tank 51 while branched from the compressed air branch piping 233 for transferring night soil into two, and a quantitative tank inner pressure application piping 235. Closing valves v11 and v12 are provided in the night soil tank inner pressure application piping 234 and quantitative tank inner pressure application piping 235. The other piping of branch pipes which are branched from the piping 232 for cleaning air into two is a compressed air branch piping 236 for cleaning catalysts, described later, and a closing valve v13 is provided in the compressed air branch piping 236 for cleaning catalysts. If the closing valve v13 of the other branch piping of the compressed air branch piping 236 for cleaning catalysts is closed, compressed air is caused to flow to the compressed air branch piping 233 for transferring night soil formed of one branch piping of the piping 232 for cleaning air. The closing valves v11 and v12 of the night soil tank inner pressure application piping 234 and quantitative tank inner pressure application piping 235 which are branched from the compressed air branch piping 233 for transferring night soil are subjected to opening and closing operation while they are switched over. That is, in the case where night soil W is transferred from the night soil tank 5 to the quantitative tank 51, the closing valve v11 of the night soil tank inner pressure application piping 234 is opened, and closing valve v12 of the quantitative tank inner pressure application piping 235 is closed. As a result, the interior of the night soil tank 5 is pressurized so that night soil W is transferred from the night soil tank 5 to the quantitative tank 51. On the other hand, opposite to the above case, in the case where the closing valve v11 of the night soil tank inner pressure application piping 234 is closed and the closing valve v12 of the quantitative tank inner pressure application piping 235 is opened, the interior of the quantitative tank 51 is pressurized so that night soil W is transferred from the quantitative tank 51 to the drying cauldron 41.

The second air system piping is an odor discharge system piping comprising apparatuses containing an odor generated from night soil, namely, the night soil tank 5 for storing night soil, quantitative tank 51, and drying cauldron 41 connected to the preheating section 71 and catalyst section 81 serving as deodorizing apparatus whereby the odor is removed to be discharged to the open air side. The odor discharge system piping is structured that the night soil tank odor discharge system piping 241 is connected to the night soil tank 5 so as to suck the odor inside the night soil tank 5 toward the outside. A closing valve v14 is provided in the night soil tank odor discharge system piping 241, and it is set to be always opened, and to be closed only in the case where compressed air from the compressor 135 is introduced into the quantitative tank 51 at the time of transferring the night soil of the quantitative tank 51. Further, quantitative tank odor discharge piping 16 is connected to the quantitative tank 51 so as to suck the odor inside the quantitative tank 51 toward the outside. The closing valve v3 is also provided in the quantitative tank odor discharge piping 16, and it is set to be always opened, and to be closed only in the case where compressed air from the compressor 135 is introduced into the drying cauldron 41 at the time of transferring the night soil to the drying cauldron 41. Still further, the discharge air piping 121 is connected to the drying cauldron 41 so as to suck the odor inside the drying cauldron 41 toward the outside of the tank. The closing valve v7 is provided also in the discharge air piping 121 and it is set to be always opened, and closed only in the case where dust is captured by the dust collection section 26 due to air circulation between the drying cauldron 41 and dust collection section 26 when the dust collection section 26 is driven. The discharge air piping 121 from the drying cauldron 41 is connected to the discharge air introduction pipe 75 of the preheating section 71. Further, the night soil tank odor discharge system piping 241 and quantitative tank odor discharge piping 16 are united with each other, and it becomes a single pipe extended from the united portion, and further, it is united with the discharge air piping (discharge air pipe) 121. In such a manner, the night soil tank odor discharge system piping 241, quantitative tank odor discharge piping 16 and discharge air piping 121 are all united with one another, which is connected to the discharge air introduction pipe 75 of the preheating section 71. The preheating section 71 has a function to suck air in the discharge air piping 121 by always heating air, as set forth above. Further, the discharge air piping 207 for discharging air to the open air side is provided in the catalyst section 81 communicating with the preheating section 71, and the ejector 210 using the fan 208 is provided at the discharge air exit of the discharge air piping 207, whereby a compulsive air discharging operation directing from the catalyst section 81 to the open air side is implemented by the ejector 210. Accordingly, air containing the odor inside the discharge air piping 121 and air containing the odor inside the night soil tank odor discharge system piping 241 connected to the discharge air piping 121 and air inside the quantitative tank odor discharge piping 16 are sucked toward the preheating section 71, respectively, so that elements of odor are adsorbed in the catalyst section 81 to become odorless, then it is discharged to the open side.

The third air system piping is a dust collection air system piping comprising the introduction pipe 154 and discharge pipe 158 respectively connecting between the drying cauldron 41 and collection box 152 of the dust collection section 26. The dust collection air system piping is provided, as shown in FIG. 14, for causing air to be circulated in the collection box 152 by the fan 174 through the introduction pipe 154 and discharge pipe 158, respectively connected to the drying cauldron 41, so as to collect rubbish in the dust bag 155 inside the collection box 152. The closing valves v4 and v5 are respectively provided in the introduction pipe 154 and discharge pipe 158 and they are subjected to opening and closing control at the same time. That is, the closing valves v4 and v5 are opened when a dust collecting step is implemented upon completion of night soil drying treatment in the drying cauldron drying cauldron 41, and they are closed before or after the dust collecting step is implemented. The fourth air system piping is an air system piping for wind pressure cleaning comprising the compressor 135, air tank 136 and catalyst section 81 which are connected with each other wherein air containing the odor is rendered odorless in the catalyst section 81, then foreign objects adhered onto the catalyst section 81 is blown off from the surfaces of catalysts by a wind pressure. The air system piping for wind pressure cleaning comprises the piping 232 for cleaning air connected to the compressor 135 for causing compressed air discharged from the compressor 135 to flow and the compressed air branch piping 233 which is one branch branched from the piping 232 for cleaning air into two. The compressed air branch piping 236 for cleaning catalysts is connected to the air tank 136 so as to fill compressed air inside the air tank 136. Meanwhile, the closing valve v13 is provided in the compressed air branch piping 236 for cleaning catalysts, described above. Further, the discharge pipe 138 for instantaneously discharging compressed air to the catalyst section 81 to blow off foreign objects adhered onto the catalyst section 81 by a wind pressure is provided between the air tank 136 and catalyst section 81. A closing valve v15 is provided in the discharge pipe 138, and is subjected to opening and closing operation by the control section.

Structure of control section:

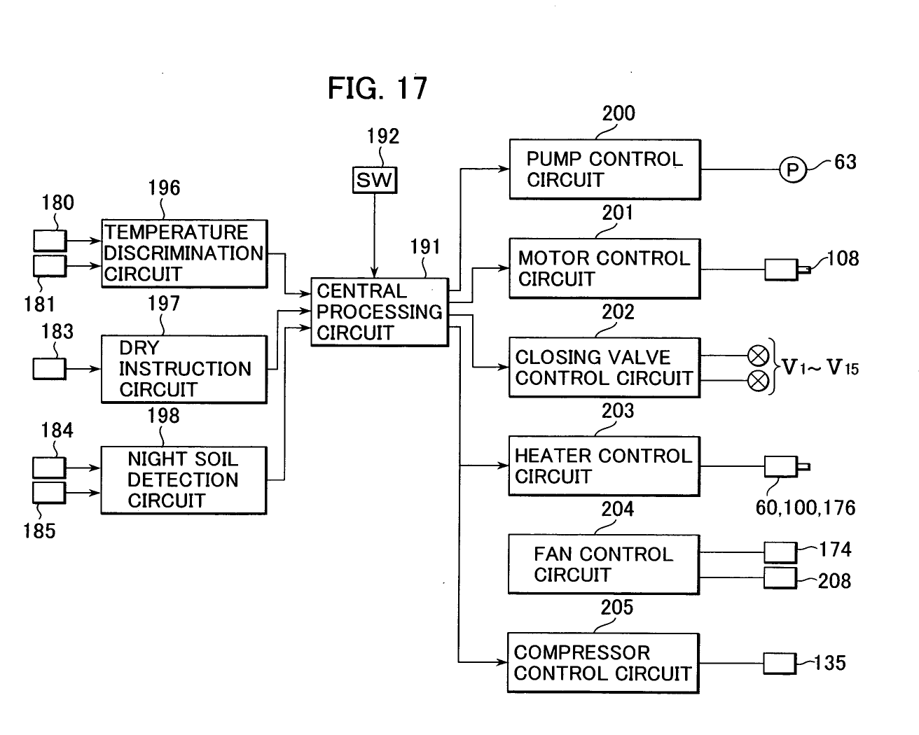

A structure of an electric system for automatically controlling the entire night soil treatment apparatus 11 according to the present embodiment will be next now described with reference to a block diagram of FIG. 17. The entire night soil treatment apparatus 11 is concentrically controlled by a central processing circuit 191, and the central processing circuit 191 is comprised of a CPU (micro processor, a central arithmetic element and so forth), a non-volatile memory (ROM) for storing a program, and so forth. A predetermined procedure is stored in the non-volatile memory and it can discriminate a given condition to operate the night soil treatment apparatus 11 in an optimum condition, and also automatically stop a treating operation while self discriminating failure or inadequate condition.

Signal system inputted to the central processing circuit 191:

A temperature sensor 180 for detecting the change of temperature is provided in the drying cauldron 41 and an output signal of temperature from the temperature sensor 180 is inputted to a temperature discrimination circuit 196. Further, a temperature sensor 181 is provided in the exit of the catalyst section 81, and an output signal of temperature from the temperature sensor 181 is also inputted to the temperature discrimination circuit 196. A discrimination signal from the temperature discrimination circuit 196 is inputted to the central processing circuit 191. An output signal from an operation instruction switch 183 is inputted to a dry instruction circuit 197, and a discrimination signal from the dry instruction circuit 197 is inputted to the central processing circuit 191 (the operation instruction switch 183 is manually operated by an operator, and not automatically operated). Further, output signals from the night soil sensor or float sensor 184 provided in the night soil tank 5 and float sensor 185 provided in the quantitative tank 51 are inputted to a night soil detection circuit 198, respectively, and a discrimination signal from the night soil detection circuit 198 is inputted to the central processing circuit 191. Meanwhile, an output of a power supply switch 192 for starting operation of the entire night soil treatment apparatus 11 is also inputted to the central processing circuit 191.

Signal system outputted from the central processing circuit 191:

The central processing circuit 191 compares various input signals with conditions stored as a program in advance inside thereof, thereby operating various apparatuses provided in the night soil treatment apparatus 11 on the basis of the result of discrimination. Accordingly, it is possible to output a plurality of control signals from the central processing circuit 191. A plurality of independent control signals are outputted from the central processing circuit 191, and respective control signals are inputted to a pump control circuit 200, a motor control circuit 201, a closing valve control circuit 202, a heater control circuit 203, a fan control circuit 204, a compressor control circuit 205.

The high pressure water generation pump 63 is connected to an output of the pump control circuit 200 and the motor 108 of the drying cauldron 41 is connected to an output of the motor control circuit 201. Further, the closing valves v1, . . . v15 are connected to an output of closing valve control circuit 202, while the heater 60 of the drying cauldron 41, the preheating heater 176 of the preheating section 71 and the catalyst heater 100 of the catalyst section 81 are connected to an output of the heater control circuit 203, respectively, and the fan 174 of the dust collection section 26 and the fan 208 of the catalyst section 81 are connected to the fan control circuit 204, respectively. Still further, the compressor 135 is connected to the compressor control circuit 205.

Night soil drying step:

Non-operation state

In a state where the night soil treatment apparatus 11 is put in storage in a warehouse and so forth, or in a state where although the night soil treatment apparatus 11 is installed on an event site, a construction site, disaster site and so forth, it is not used at night or on holiday and so forth, the power supply switch 192 is turned off (namely, the circuit is not turned on), respective apparatuses constituting the night soil treatment apparatus 11 maintains a stopping state. If the power supply switch 192 is set to be turned off, the central processing circuit 191 maintains a stopping state in response to a signal outputted from the power supply switch 192, and it supplies a stopping signal to the pump control circuit 200, motor control circuit 201, closing valve control circuit 202, heater control circuit 203, fan control circuit 204 and compressor control circuit 205. In the stopping state, the pump control circuit 200 stops the operation of the high pressure water generation pump 63 while the motor control circuit 201 stops the motor 108, the fan control circuit 204 stops the fans 174 and 208, and the compressor control circuit 205 stops the compressor 135. The closing valve control circuit 202 closes the closing valves v1 to v15, and the heater control circuit 203 does not supply electricity to heaters 60, 100 and 176.

Installment of the night soil drying treatment apparatus:

When excreta is treated by installing the night soil drying treatment apparatus on an event site, a construction site, a disaster site and so forth, the temporary toilet 1 and night soil treatment apparatus 11 shown in FIG. 1 are carried out from the warehouse and are transferred to an intended installing place by a truck and so forth. If the temporary toilet 1 and night soil treatment apparatus 11 are disposed adjacent to each other in the installing place, both the temporary toilet 1 and night soil treatment apparatus 11 are assembled. Thereafter, a commercial power or power from an electric accumulator is connected to the night soil treatment apparatus 11 so as to be ready for supplying power to respective mechanisms inside the night soil treatment apparatus 11. In such a manner, a set of night soil drying treatment apparatus can be arranged, thereby completing preparation for coping with excreting behavior of people participating in the event, people working in the construction site, or people in the disaster site.

Standby by turning on a power supply:

In the case where the night soil drying treatment apparatus is temporarily provided on the construction site, event site, disaster site and so forth so as to be temporarily used, the temporary toilet 1 and respective mechanisms housed in the night soil treatment apparatus 11 are started up, thereby proceeding to a standby state so as to immediately treat night soil excreted to the toilet stool 6. The power supply switch 192 provided in the night soil treatment apparatus 11 is turned on (power supply is turned on) so as to set the night soil drying treatment apparatus in a standby state. When the power supply switch 192 is turned on, the night soil treatment apparatus starts an operation so that the night soil treatment apparatus is rendered in a state to start treatment of night soil. A signal from the power supply switch 192 generated when the power supply switch 192 is turned on is transmitted to the central processing circuit 191, so that the central processing circuit 191 switches over the states of the pump control circuit 200, motor control circuit 201, closing valve control circuit 202, heater control circuit 203, fan control circuit 204 and compressor control circuit 205 to operable states. However, unless there is operation for turning on the operation instruction switch 183 (a behavior for starting operation manually), the pump control circuit 200, motor control circuit 201, closing valve control circuit 202, heater control circuit 203, fan control circuit 204 and compressor control circuit 205 do not at all operate. It is because in the case where night soil to be subjected to drying treatment is not stored in the night soil tank 5, the night soil drying treatment apparatus is not required to operate.

Use of the temporary toilet 1 by a utilizer:

If the power supply switch 192 is turned on, both the mechanisms of the temporary toilet 1 and night soil treatment apparatus 11 are rendered in a standby state, so that the utilizer can use the night soil drying treatment apparatus at any time. When the temporary toilet 1 in the standby state is used, the utilizer pulls the door open, not shown, to enter the house. If the utilizer enters the interior of the house, he or she excretes night soil toward the toilet stool 6. Accordingly, night soil is thrown in the interior of the night soil tank 5 as it is through the lower opening of the toilet stool 6, then night soil is stored in the night soil tank 5 in a state as it is. These mechanisms have the same function as a tank type temporary toilet which has been conventionally used.

Start of evaporating drying treatment:

In the case where the temporary toilet 1 is used to some extent and the storage quantity of night soil W inside the night soil tank 5 increases to reach a set quantity detected by the float sensor 184, a transfer and evaporating drying treatment of night soil W are started automatically in response to the instruction from the control device. The start of this treatment causes night soil W to flow in the direction from the night soil tank 5 to the quantitative tank 51 and night soil treatment apparatus 11 so that the evaporating drying treatment is started.

Throwing of night soil W in the quantitative tank 51 and drying treatment:

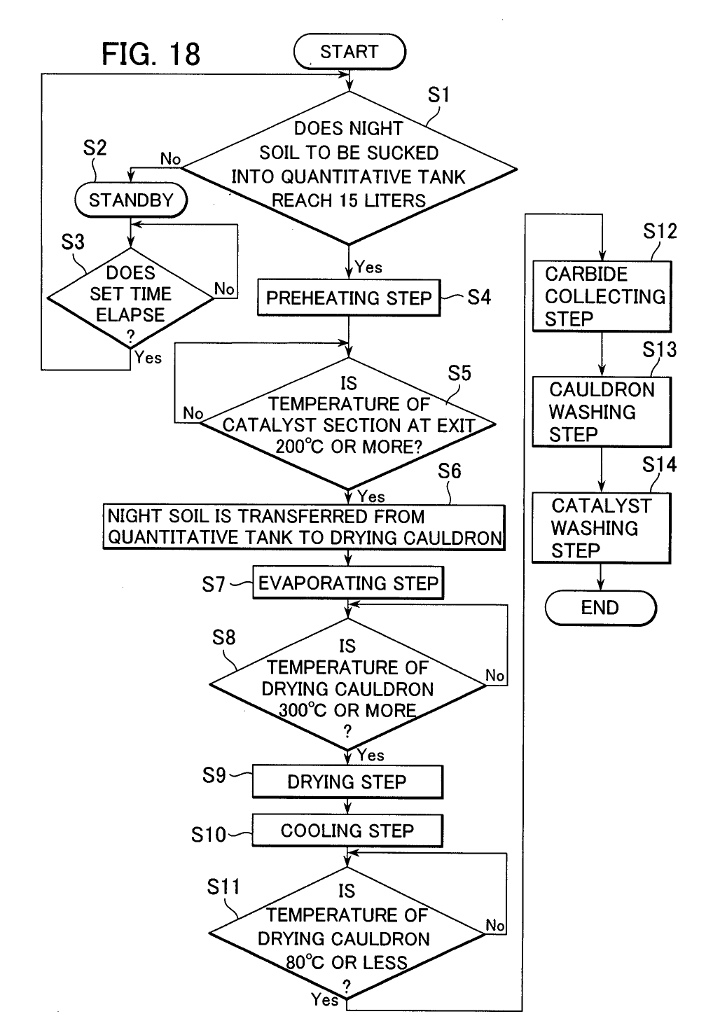

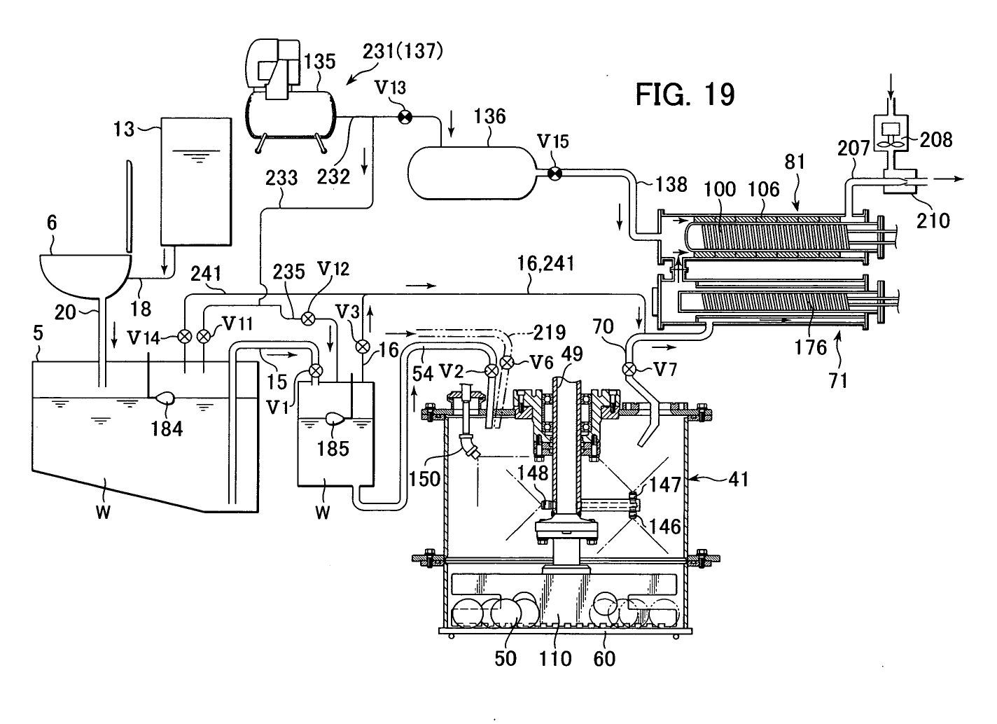

FIG. 18 is a flow chart showing the driving of the night soil treatment apparatus according to the present embodiment. FIG. 18 shows in detail a transferring step (S1 to S3) for transferring night soil W from the night soil tank 5 to the quantitative tank 51 after the power supply switch 192 is turned on, a transferring step (S4 to S6) for transferring night soil W from the quantitative tank 51 to the drying cauldron 41 and a drying treatment step (S7 to S11), and also roughly shows a step of collecting carbide (S12), a step of washing the drying cauldron 41 (S13) and a step of washing the catalysts (S14). Further, FIG. 19 shows a structure for explaining the procedure shown in FIG. 18 while comparing with concrete structure of the apparatus. The throwing of night soil W in the quantitative tank 51 and the drying treatment are described hereinafter with reference to FIG. 18 and FIG. 19.

As shown in FIG. 18, when the power supply is turned on (START), it is decided as to whether the quantity of night soil stored in the night soil tank 5 reaches a given quantity (e.g. 15 liters) to be transferred to the quantitative tank 51 (S1). Meanwhile, the preheating section 71 and catalyst section 81 are heated to a given temperature at the same when the power supply is turned on so that air in the apparatus system is sucked, thereby establishing a system to absorb the odor. In the case where the quantity of night soil stored in the night soil tank 5 does not reach a given quantity to be transferred to the quantitative tank 51, an operation is in a standby for a given time (S2) while if a standby time exceeds a set time, the operation reverts to step S1 so that the decision of the quantity of night soil in the night soil tank 5 is repeated. If the quantity of night soil inside the night soil tank 5 reaches a given quantity (e.g. 15 liters) (S1, YES), night soil is transferred from the night soil tank 5 to the quantitative tank 51. Subsequently, a preheating step is started (S4), so that temperatures at exits of the preheating section 71 and catalyst section 81 are set up to a given temperature, (e.g. 200° C. or more) (S5). Then, night soil is transferred from the quantitative tank 51 to the drying cauldron 41 (S6).

A concrete operation at the time of transferring night soil from the night soil tank 5 to the quantitative tank 51 and drying cauldron 41 is described with reference to FIG. 19. In the steps of S1 to S3, the closing valve v13 of the compressed air branch piping 236 for cleaning catalysts provided in the piping 232 for cleaning air connected to the compressor 135 is closed, as shown in FIG. 19. In the case where it is decided that the quantity of night soil W stored inside the night soil tank 5 at the toilet stool 6 side reaches a set quantity of night soil to be subjected to drying treatment, the closing valve v1 provided in the night soil transfer pipe 15 is opened, and when the compressor 135 is actuated, the closing valve v11 of the night soil tank inner pressure application piping 234 is opened and compressed air is introduced into to the night soil tank 5 through the piping 232 for cleaning air, compressed air branch piping 233 for transferring night soil and night soil tank inner pressure application piping 234, so that a pressurized force thereof acts on night soil inside the night soil tank 5. In this case, the closing valve v14 of the night soil tank odor discharge system piping 241 is closed. The closing valve v13 of the quantitative tank odor discharge piping 16 is opened. Then, the set quantity of night soil as one load of night soil to be subjected to drying treatment in the drying cauldron 41 is transferred to the interior of the quantitative tank 51 through the night soil transfer pipe 15 by air pressure from the compressor 135. Then, if the night soil sensor 185 inside the quantitative tank 51 detects that the set quantity of night soil is stored inside the quantitative tank 51, the closing valve v1 provided in the night soil transfer pipe 15 is closed. At the same time, the closing valve v11 of the night soil tank inner pressure application piping 234 is closed and the closing valve v14 of the night soil tank odor discharge system piping 241 is opened. Then, if an instruction representing transfer of night soil to the drying cauldron 41 is outputted in Step 6, the closing valve v12 of the quantitative tank inner pressure application piping 235 is opened so that compressed air from the compressor 135 is supplied to the quantitative tank 51. At the same time, the closing valve v3 of the quantitative tank odor discharge piping 16 is closed. The closing valve v12 of the night soil transfer pipe 15 is kept closed. Further, the closing valve v2 of the night soil drying transfer pipe 54 is opened. Accordingly, the quantity of night soil as set for one load of night soil to be subjected to drying treatment inside the drying cauldron 41 is transferred to the interior of the drying cauldron 41 by the air pressure from the compressor 135 through the night soil drying transfer pipe 54.

Then, as shown in FIG. 18, night soil drying treatment in the drying cauldron 41 is implemented in the procedure in steps S7 to S11. The drying cauldron 41 is first heated by the heater 60 so that an evaporating step (S7) for evaporating moisture of night soil W is implemented. In the evaporating step, an evaporation operation is implemented when the temperature of the drying cauldron 41 exceeds a given level (e.g. 300° C. or more). In the evaporating step, the closing valve v7 of the discharge air piping (discharge air pipe) 121 of the drying cauldron 41 is opened so that generated vapor flows from the discharge air piping 121 to the preheating section 71 to be preheated, then it enters the catalyst section 81 where the odor is removed by the catalysts 106, then discharged to the open air side from the exit of the discharge air piping 207 owing to the suction operation of the ejector 210. In this case, piping other than the discharge air piping 121 connected to the drying cauldron 41 are all closed. Upon completion of generation of vapor, the operation goes to a drying step (S9), where moisture of night soil is completely eliminated and night soil becomes carbide as a solid material. During the drying step, the stirring shaft 49 is rotated in the drying cauldron 41 so that carbide is powdered by the balls 50 for stirring purpose serving as the accumulators. The foregoing steps is repeated by several times until the set number, where night soil inside the night soil tank 5 goes substantially out of existence, is completed. If set drying treatment is completed, heating of the heater 60 is stopped so that the closing valve v6 of the open air introduction pipe 219 is opened to introduce the open air into the drying cauldron 41 and the operation is in a cooling step (S10), then the temperature of the drying cauldron 41 is cooled up to a set temperature (e.g. 80° C. or less) (S11). After the temperature of the drying cauldron 41 is reduced to the set temperature or less, a dust collecting step serving as carbide collecting step is implemented (S12).

Dust Collecting Step:

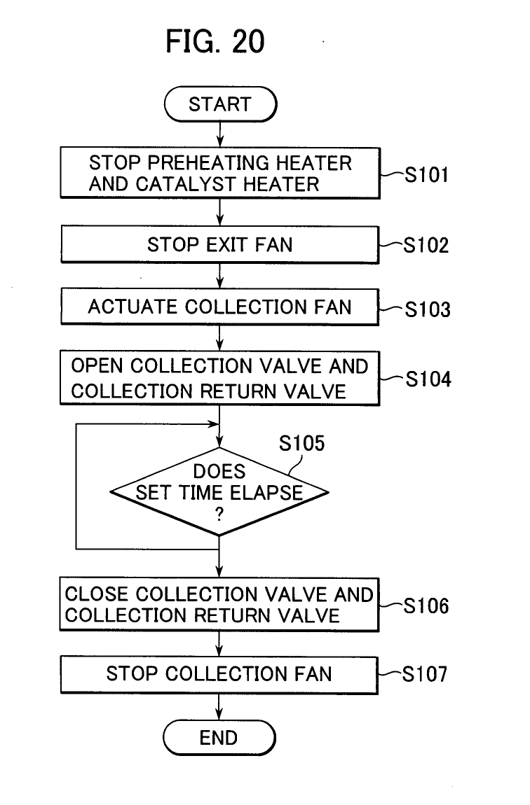

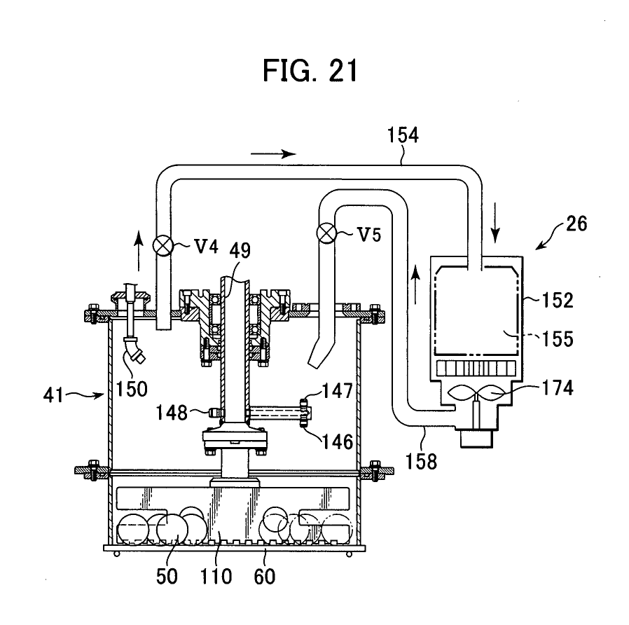

FIG. 20 shows a procedure of a dust collecting step, and FIG. 21 shows the structures of the apparatuses in the dust collecting step. As shown in FIG. 21, the air system piping comprises the introduction piping 154 and the discharge pipe 158 for connecting the drying cauldron 41 and collection box 152, respectively. The dust collection section 26 is structured to capture dust from air to cause only clean air to flow out to the outside. The air system piping for collecting dust is structured, as shown in FIG. 14, to cause air to circulate in the collection box 152 by the fan 174 via the introduction pipe 154 and discharge pipe 158 connected to the drying cauldron 41 so as to collect dust in the dust bag 155 provided inside the collection box 152. The valves v4, v5 are provided in the introduction pipe 154 and discharge pipe 158, and they are subjected to opening and closing control at the same time.

In the dust collecting step, the preheating heater 176 and catalyst heater 100 are first stopped (S101). Then the fan (exit fan) 208 of the catalyst section 81 is stopped (S102) to actuate the fan (collection fan) 174 of the dust collection section 26 (S103). Thereafter, the closing valves (collection valve and collection return valve) v4, v5 are opened (S104). As a result, air flown in the interior of the dust bag 155 from the introduction pipe 154 passes through the film surface of the dust bag 155 so that dust mixed in air is captured by the film surface of the dust bag 155, and only clean air flows the inner space of the collection box 152. This step is implemented for a set time (S105), then the closing valves v4, v5 are closed (S106) and the collection fan 174 is stopped (S107). With this procedure, air is circulated in the collection box 152 by the fan collection 174 via the introduction pipe 154 and discharge pipe 158 connected to the drying cauldron 41 so that dust can be collected in the dust bag 155 inside the collection box 152.

Drying Cauldron Cleaning Step:

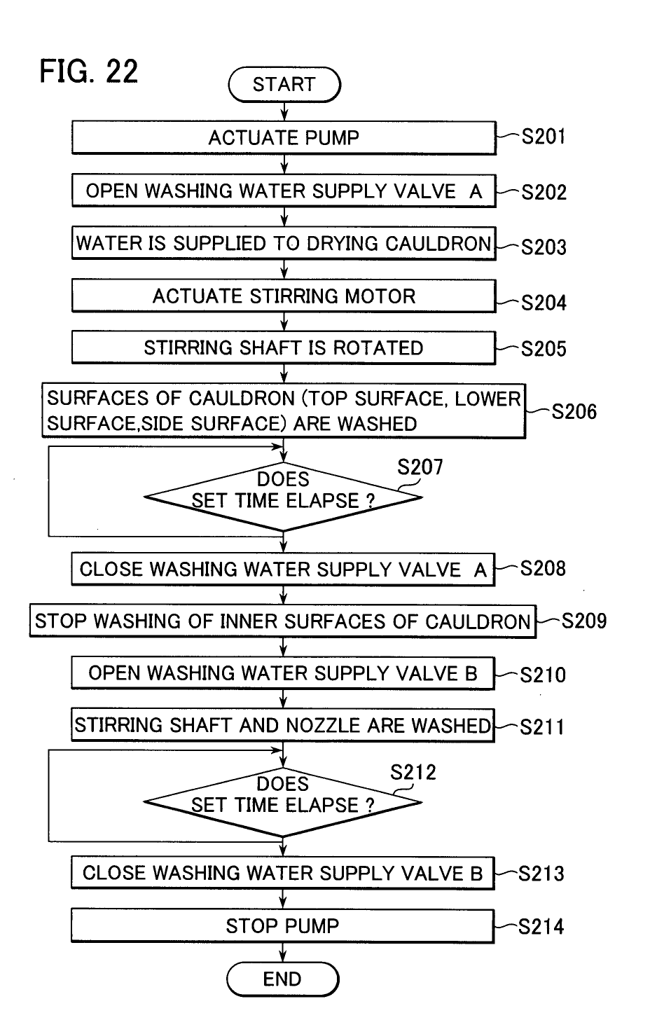

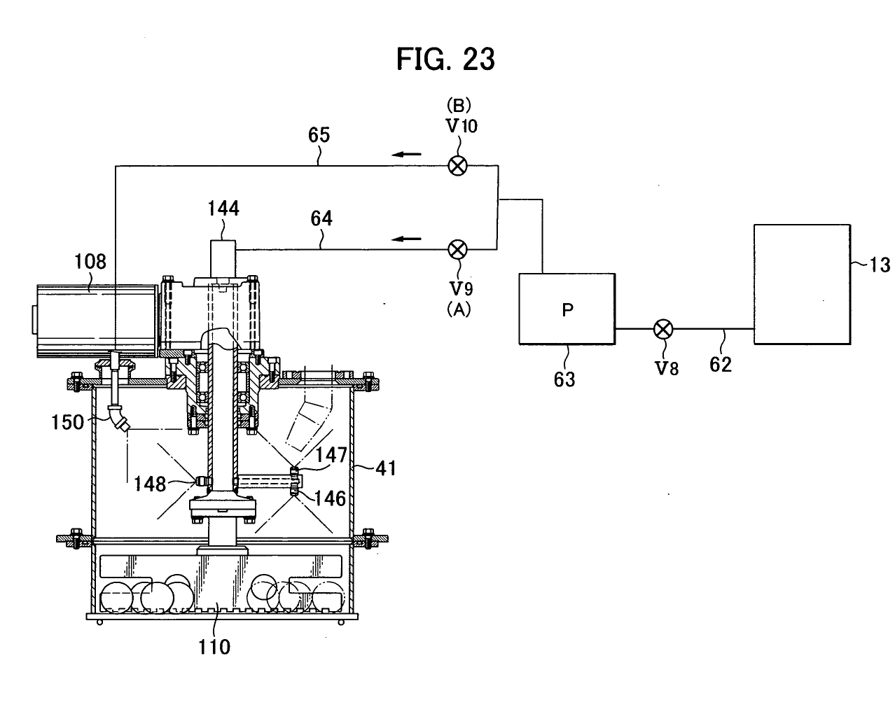

FIG. 22 shows a procedure of the drying cauldron cleaning step, and FIG. 23 shows the structures of the apparatuses in the drying cauldron cleaning step. The washing water supply system is first described with reference to FIG. 23. The cauldron washing pipe 62 and closing valve v8 are connected to the water tank 13 serving as the water supply source, and the high pressure water generation pump 63 is provided in the cauldron washing pipe 62. The cauldron washing pipe 62 is branched at the downstream side of the high pressure water generation pump 63, and one branch pipe 64 is connected to the stirring shaft 49 via the rotary joint 144 provided at the central position of the cauldron lid 59 of the drying cauldron 41. The downward directed washing nozzles 146, upward directed cauldron inner ceiling washing nozzle 147 and sideward directed cauldron inner peripheral wall washing nozzle 148 are provided at the lower end of the stirring shaft 49. The closing valve v9 (hereinafter refereed to as valve A) is provided in one branch pipe 64 of the cauldron washing pipe 62. The other branch pipe 65 branched from the cauldron washing pipe 62 at the downstream side of the cauldron washing pipe 62 is connected to the stirring blade washing nozzle 150 provided at the side of the interior of the drying cauldron 41. The closing valve v10 (hereinafter referred to as valve B) is provided in the other branch pipe 65.

The washing operation is next described with reference to FIG. 22. First, the high pressure water generation pump 63 is actuated (S201) and the valve A is opened (S202). At this time, the valve B is closed. Further, the motor 108 is actuated (S204). As a result, the stirring shaft 49 starts rotation (S205). Washing water supplied from the high pressure water generation pump 63 flows into one branch pipe 64, then flows into the stirring shaft 49 via the rotary joint 144, thereafter jetted from the washing nozzles 146, 147 and 148 (S203). Washing water is jetted downward, upward, and sideward relative to the drying cauldron 41 in a fan-shape, and high pressure water is supplied to the interior of the drying cauldron 41 so that the upper surface, lower surface and side surface of the drying cauldron 41 are washed by jetted water (S206). Washing operation is implemented until a set time (S207). Meanwhile, in the washing operation by jetting water from the nozzles provided on the stirring shaft 49, foreign objects which were washed from the interior of the drying cauldron 41 are adhered onto the stirring blade 110 because the stirring blade 110 provided on the inner bottom portion of the drying cauldron 41 is located at the lower position. Accordingly, the valve A is closed (S208) after the elapse of set time of washing operation so as to once stop the washing of the interior of the drying cauldron 41 (S209).

Thereafter, the valve B is opened (S210) to wash the stirring shaft 49 and nozzles (S211). That is, washing water supplied from the high pressure water generation pump 63 is switched over to the branch pipe 65, thereby supplying washing water to the stirring blade washing nozzle 150 provided at the side portion of the interior of the drying cauldron 41. By this, high pressure water is jetted from the sideway of the interior of the drying cauldron 41 in a fan-shape so that the stirring blade 110 provided at the inner bottom of the drying cauldron 41, bolls 50 serving as the heat accumulators, washing nozzles 146, 147 and 148, and so forth are washed by jetted washing water. The washing operation is implemented during a set time (S212), then the valve B is closed (S213) and the high pressure water generation pump 63 is stopped (S214).

With the above-mentioned operations, water is jetted toward the interior of the drying cauldron 41 upon completion of night soil treatment to wash the interior of the drying cauldron 41, thereby removing adherents onto the drying cauldron 41, so that carbide inside the drying cauldron 41 can be substantially completely removed. Accordingly, it is possible to prevent carbide from being remained inside the drying cauldron 41 to thereby clean the ceiling portion, peripheral wall portion, bottom portion and so forth of the interior of the drying cauldron 41, so that resistance to rotation of the stirring blade 110 is not generated when the drying cauldron 41 is used, and generation of abnormal noise caused by friction and generation of trouble of the motor 108 and so forth can be prevented. Further, since the washing mechanism is comprised of the water tank 13 for washing the toilet stool 6, cauldron washing pipe 62 for introducing water from the water tank 13 into the drying cauldron 41, high pressure water generation pump 63 provided in the cauldron washing pipe 62 and jet nozzles 146, 147, 148 and 150 provided at the tip end of the cauldron washing pipe 62 for jetting high pressure water toward the interior of the drying cauldron 41, the structure becomes simplified. Further, the jet nozzles 146, 147 and 148 are provided on the stirring shaft 49 which is rotated in the drying cauldron 41 and washing water is jetted toward the interior of the drying cauldron 41 while the stirring shaft 49 is rotated, the drying cauldron 41 can be washed throughout the interior thereof. Further, since the water washing pipe 18 is divided into plural numbers and washing water from the water tank 13 can be jetted toward the different positions of the interior of the drying cauldron 41 by switching over the valves, thereby individually washing the respective different positions.

Catalyst washing step:

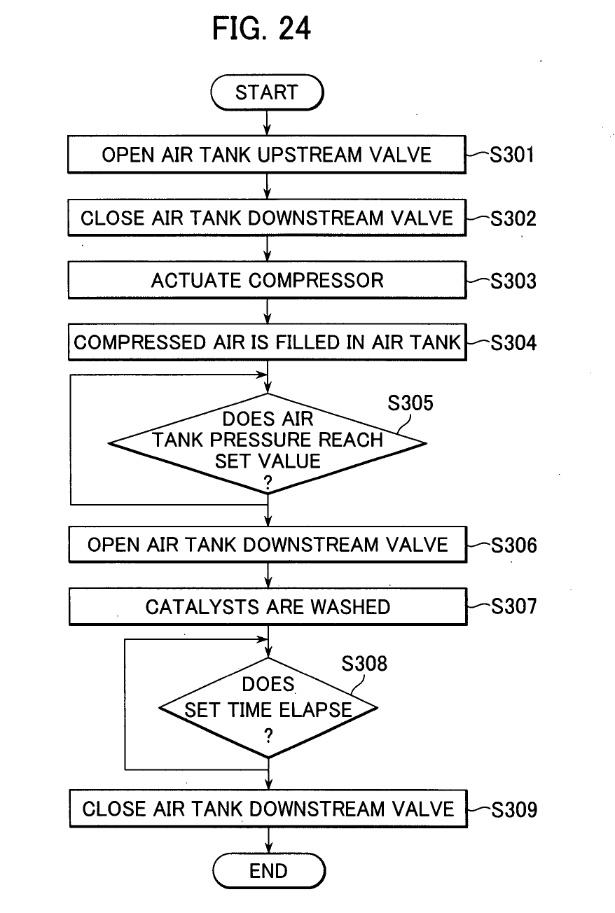

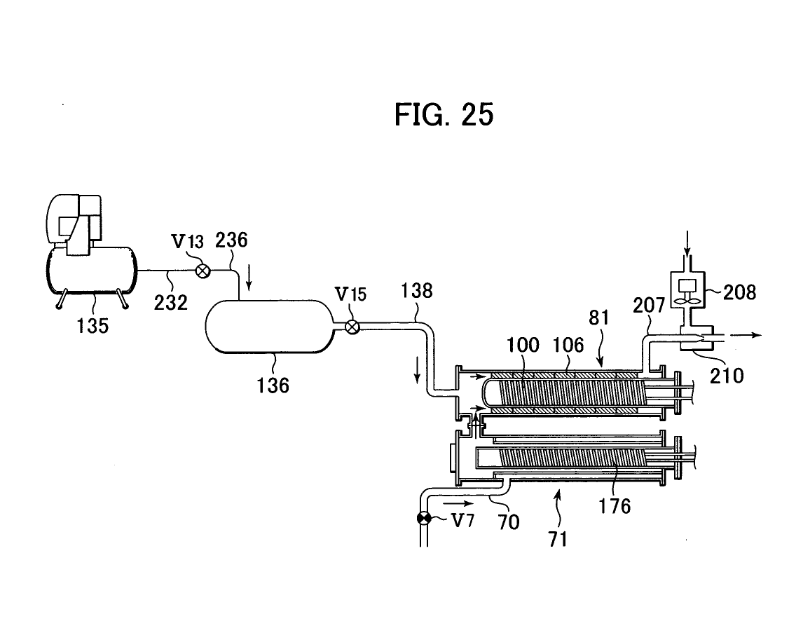

FIG. 24 shows a procedure of a catalyst washing step and FIG. 25 shows structures of the apparatuses in the catalyst washing step. A deodorizing apparatus has been conventionally provided in a deodorizing case for extinguishing the odor discharged from the drying cauldron 41. The deodorizing apparatus houses various catalysts in the deodorizing case and captures deodorizing components through discharge air which passes through the catalysts. However, it is impossible to completely collect the entire amount of powdered foreign objects in which the deodorizing components are contained, and the powdered foreign objects are adhered and accumulated little by little onto the interior of the catalysts while deodorizing apparatus is operated. Accordingly, unless the interior of the catalyst case is not cleaned periodically, the flow of an odor is impaired by the adhered foreign objects, so that the inherent deodorizing function can not be maintained. Further, since the working for taking out the catalysts so as to clean the catalysts has to be implemented after the heater has been sufficiently cooled, it requires cooling time, and if the cleaning is implemented without confirming the cooling, there is a likelihood of involving risk such as burn injury and so forth. Further, since the interior of the catalysts is manufactured with appropriate roughness which is neither so coarse nor fine, the microscopic foreign objects which are not captured reach the catalyst section 81 to be adhered onto the surfaces of catalysts. Accordingly, it is necessary to clean the catalysts periodically when taking out them, but the working for attaching and detaching the catalysts which are contained in the catalyst case with no space therebetween is very difficult. Further, there is a likelihood that the catalysts are damaged by heating and vibration and the catalysts are clogged with broken pieces.

To that end, according to the present embodiment, as shown in FIG. 25, there is provided with a wind cleaning mechanism for supplying compressed air to the catalyst section 81 upon completion of drying treatment so as to blow off the adherents onto the surfaces of the catalysts 106. The wind cleaning mechanism comprises the compressor 135 and air tank 136 for storing therein compressed air supplied from the compressor 135, and instantaneously jetting compressed air toward the catalyst section 81. Further, the wind cleaning mechanism is set to implement cleaning treatment every time one load of night soil is subjected to drying treatment in the drying cauldron 41.

More in detail, as shown in FIG. 25 and FIG. 16, the air tank 136 is connected to the compressor 135 via the compressed air branch piping 236 for cleaning catalysts which has been branched from the piping 232 for cleaning air. The closing valve v13 is provided in the compressed air branch piping 236 for cleaning catalysts. The air tank 136 and catalyst section 81 are connected to each other by the discharge pipe 138 so that compressed air is instantaneously discharged from the air tank 136 to the catalyst section 81, thereby blowing off the foreign objects adhered onto the catalyst section 81 by a wind pressure. The closing valve v15 is provided in the discharge pipe 138, which is subjected to opening and closing operation by the control section.

This operation is now described with reference to FIG. 24. First, the closing valve v13 at the upstream side of the air tank 136 is opened (S301), while the closing valve v15 at the downstream side of the air tank 136 is closed (S302). In this state, the compressor 135 is actuated (S303). When the compressor 135 is actuated, compressed air is filled in the air tank 136 (S304). If a pressure of compressed air (air tank pressure) filled in the air tank 136 reaches a set value (S305), the closing valve v15 at the downstream side of the air tank 136 is instantaneously opened (S306). With this operation, high-pressure air is instantaneously supplied to the catalyst section 81 via the discharge pipe 138. Accordingly, carbide contained in air sucked from the drying cauldron 41 side, and also contained in air and vapor which are generated during night soil treatment or washing treatment of the interior of the drying cauldron 41 and flow to the catalyst section 81, carbide contained in vapor and so forth adhered onto the surfaces of catalysts, and other foreign objects are instantaneously separated and removed from the surfaces of catalysts, then discharged toward the open air side through the discharge air piping 207 (S307). The catalyst cleaning is implemented for a set time, and it is completed upon elapse of the set time (S308). Thereafter, the closing valve v15 at the downstream side of the air tank 136 is closed (S309).

With such a structure and operation, the odor in discharge air upon completion of drying treatment is removed and the adherents onto the surfaces of catalysts are blown off by the wind pressure to automatically wash the surfaces of catalysts so that the surfaces of the metallic catalysts and so forth can be always kept clean, and a function to suck the odor can be maintained over a long term. Further, since as the wind pressure cleaning mechanism is structured that compressed air supplied from the compressor 135 is stored in the air tank 136 and it is instantaneously jetted by the catalyst mechanism at the time of cleaning, the catalyst can be efficiently cleaned in a short time with a simple structure. Still further, since the wind pressure cleaning mechanism is set to implement cleaning treatment every time one load of night soil is subjected to drying treatment in the drying cauldron 41, it is possible to remove the objects adhered onto the catalysts 106 immediately after they are adhered onto the catalysts 106, and to peel off the adherents with ease before they are solidified.

According to the embodiment set forth above, washing water is jetted toward the interior of the drying cauldron 41 every time one cycle of night soil dying treatment is completed by providing the washing mechanism for removing adherents onto the interior of the drying cauldron 41, thereby removing the adherents onto the interior of the drying cauldron 41 by automatically washing the interior of the drying cauldron 41, so that it is possible to prevent carbide and so forth from being adhered, solidified, accumulated and so forth in the interior of the drying cauldron 41 every time drying treatment of night soil W is completed. Accordingly, since heat transfer of the heater 60 of the drying cauldron 41 caused by the accumulation of carbide and so forth is not hindered, it is possible to smoothly implement treatment of the next cycle and succeeding treatment. For example, even in the structure for implementing control based on the temperature of the drying cauldron 41, the next treatment step can be proceeded under the complete condition so that the structure can be continuously used for a long term. Since carbide is prevented from adhered onto the interior upper surface of the drying cauldron 41 and carbide collection piping inlet, the necessity of periodic cleaning of the interior of the drying cauldron 41 can be considerably reduced, and the frequency of cleaning of the interior of the drying cauldron 41 can be sharply reduced. Further, the problem of clogging of piping and so forth can be solved. Accordingly, the washing mechanism is almost dispensed with cleaning of the interior of the drying cauldron 41 by periodically detaching the drying cauldron 41 or top plate in order to remove carbide which has been made conventionally, the working for attaching and detaching the top late of the drying cauldron 41 by an operator, the working for scraping carbide in the drying cauldron 41, the working for collecting the scraped carbide and so forth, so that the service life of the apparatus can be lengthened while omitting many troublesome time and effort, and convenience in various installing place can be enhanced, and also time, effort and cost involved in the operation and storage of the apparatus and so forth can be reduced.

Further, since the cauldron washing pipe 62 for introducing water from the water tank 13 into the drying cauldron 41 can be used in the washing mechanism, the washing cleaning mechanism is simplified by efficiently utilizing the same water supply source as that for washing the toilet stool, and also the washing mechanism is provided with the high pressure water generation pump 63 provided in the cauldron washing pipe 62, jet nozzles 146, 147, 148 and 150 provided at the tip end of the cauldron washing pipe 62 for jetting high pressure water toward the interior of the drying cauldron 41, carbide and other various foreign objects adhered onto the interior of the drying cauldron 41 can be washed with high capacity, thereby implementing washing operation efficiently with high functionality. Still further, the jet nozzles 146, 147, 148 are provided on the stirring shaft 49 which is rotated in the drying cauldron 41, and they are structured to jet washing water toward the interior of the drying cauldron 41 while the stirring shaft 49 is rotated, so that uniform cleaning can be implemented by extensively jetting washing water toward all over the interior of the drying cauldron 41. More still further, the washing pipe is divided into plural numbers as the branch pipes 64, 65, and washing water from the water tank 13 is jetted toward the different positions of the interior of the drying cauldron 41 so as to individually implement washing by switching over valves of the branch pipes 64, 65, so that washing effect can be further enhanced by washing water from various directions. Further, it is possible to wash the stirring blade 110, various washing pipes per se and so forth, thereby preventing the jet nozzles 146, 147, 148, 150 from being clogged, thereby exerting excellent function in terms of maintenance of soundness of the apparatus.

Further, the catalyst section 81 for removing the odor in discharge air upon completion of drying treatment is provided with the wind pressure cleaning mechanism for blowing off adherents onto the surfaces of catalysts by a wind pressure while supplying compressed air after treatment, so that the wind pressure cleaning mechanism has a function to automatically clean the surfaces of catalysts. Accordingly, the surfaces of the metallic catalysts and so forth can be always kept clean, and a function to suck the odor can be maintained over a long term. Further, since the wind pressure cleaning mechanism is structured that compressed air supplied from the compressor 135 is stored in the air tank 136 to instantaneously jet compressed air toward the catalyst section 81 at the time of cleaning, the catalysts can be efficiently cleaned in a short time with a simple structure. Still further, it is possible to remove the objects adhered onto the catalysts 106 immediately after they are adhered onto the catalysts 106, and to peel off the adherents with ease before they are solidified.

As mentioned above, according to the first aspect of the invention, there is provided the water washing mechanism for removing adherents onto the drying cauldron, which mechanism jets water toward the interior of the drying cauldron every time one cycle of drying treatment of night soil is completed so as to automatically wash the drying cauldron to remove the adherents onto the drying cauldron, thereby preventing carbide and so forth from being adhered onto the drying cauldron, solidified and accumulated and so forth in the drying cauldron every time drying treatment of night soil is completed. Accordingly, it is possible to smoothly implement treatment of the next cycle and succeeding treatment of without hindrance of heat transfer from the heater inside the drying cauldron which is caused by the accumulation of carbide and so forth, for example, even in the case where the apparatus is controlled control based on the temperature of the bottom of the drying cauldron and the temperature of the heater, the next treating step can be proceeded under a complete control condition, and the apparatus can be continuously used over the long term. Further, it is possible to prevent carbide from being adhered onto the upper surface of the interior of the drying cauldron and carbide collection piping inlet, so that the necessity of cleaning of the interior of the drying cauldron at regular intervals is extremely reduced, thereby extensively reducing frequency of cleaning of the interior of the drying cauldron, and solving the problem of clogging of piping and so forth. Accordingly, cleaning of the interior of the drying cauldron or top plate at regular intervals by removing the drying cauldron or top plate so as to remove carbide, and the working for attaching and detaching the top plate of the drying cauldron by an operator, the working for scraping carbide off the drying cauldron, the working for collecting scraped carbide and so forth are almost dispensed with, so that the lifetime of the night soil drying treatment apparatus can be lengthened by omitting much time and effort involved in such troublesome workings, and convenience in various installing places can be enhanced, and also time, effort 1 and cost involved in the operation and putting in storage can be enhanced.

According to the second aspect of the invention, the washing mechanism can simplify the structure of the apparatus by using the water tank for washing the toilet stool and the washing water pipe for introducing water into the drying cauldron from the water tank, thereby efficiently utilizing the same water supply, and also since the apparatus includes the pump for generating high pressure water provided in the washing water pipe and the jet nozzle provided at the tip end of the washing water pipe for jetting high pressure water toward the interior of the drying cauldron, carbide and other various foreign objects adhered onto the interior of the drying cauldron can be flushed with a high capacity, and also the washing operation can be implemented with high function and efficiency.

According to the third aspect of the invention, since it is structured that the jet nozzles are provided on the stirring shaft which is rotated in the drying cauldron so as to jet washing toward the interior of the drying cauldron while the stirring shaft is rotated, washing water can be jetted toward the entire interior of the drying cauldron, thereby implementing uniform washing extensively.

According to the fourth aspect of the invention, since it is structured that the washing water pipe is divided into plural numbers, and washing water can be jetted toward the different positions of the interior of the drying cauldron by switching over the valves, thereby washing the drying cauldron individually, so that the washing effect can be further enhanced by washing from various directions, and washing of the respective washing water pipes per se and so forth can be implemented, thereby preventing the nozzles from being clogged to exert an excellent function in terms of maintenance of soundness and so forth of the apparatus.

According to the fifth aspect of the invention, the catalyst mechanism for removing the odor in discharge air upon completion of drying treatment is provided with the wind pressure cleaning mechanism for blowing off adherents onto the surfaces of catalysts by wind pressure generated by supplying compressed air upon completion of drying treatment so as to have the cleaning function to automatically wash the surfaces of catalysts so that the surface of the metallic catalysts and so forth can be always kept clean and the function of sucking the odor can be maintained over the long term.

According to the sixth aspect of the invention, since the compressed air supplied from the compressor is stored in the air tank as wind the pressure cleaning mechanism, thereby jetting compressed air instantaneously toward the catalyst mechanism at a cleaning time, the catalysts can be cleaned efficiently in a short time with a simple structure.

According to the seventh aspect of the invention, since the wind pressure cleaning mechanism is set to implement cleaning treatment every time one load of night soil is subjected to drying treatment in the drying cauldron, the materials adhered onto the catalysts can be removed immediately after they adhered onto the catalysts, so that the adhered materials can be peeled off before they are solidified.

1. A cauldron interior cleaning mechanism of a night soil drying treatment apparatus comprising a temporary toilet provided with a night soil tank capable of storing therein night soil and a toilet stool communicating with the night soil tank at its lower portion for causing night soil to flow down, and a night soil drying treatment mechanism comprised of a heat resistant closed drying cauldron for storing night soil therein, and a heating means for heating the drying cauldron to evaporate night soil, thereby subjecting night soil to drying treatment by evaporating night soil in the airtight drying cauldron, wherein a night soil pipe for causing night soil to fluid therein is connected between the night soil tank and the drying cauldron for causing night soil inside the night soil tank to be sucked up and thrown in the drying cauldron, and upon storing a load of night soil to be subjected to drying treatment in the drying cauldron, the drying cauldron is heated to evaporate night soil in the drying cauldron, and the heating and evaporating treatment are carried out by a set number to complete the night soil drying treatment, characterized in that the cauldron interior cleaning mechanism is comprised of a washing mechanism for jetting water to the interior of the drying cauldron to wash the interior of the drying cauldron upon completion of night soil dying treatment, thereby removing adherents onto the interior of the drying cauldron.

2. The cauldron interior cleaning mechanism according to claim 1, wherein the washing mechanism comprises a water tank for washing the toilet stool, a washing water pipe for introducing water from the water tank into the drying cauldron, a high pressure water generation pump provided in the washing water pipe, and jet nozzles provided at the tip end of the washing water pipe for jetting high pressure water toward the interior of the drying cauldron.

3. The cauldron interior cleaning mechanism according to claim 2, wherein the jet nozzles are provided on a stirring shaft rotating in the drying cauldron to jet washing water to the interior of the drying cauldron while the stirring shaft is rotated.

4. The cauldron interior cleaning mechanism according to claim 3, wherein the washing pipe is divided into plural numbers through which washing water is jetted toward different positions of the interior of the drying cauldron by switching over valves, thereby individually washing the interior of the drying cauldron.

5. A catalyst cleaning mechanism of a night soil drying treatment apparatus comprising a temporary toilet provided with a night soil tank capable of storing therein night soil and a toilet stool communicating with the night soil tank at its lower portion for causing night soil to flow down, and a night soil drying treatment mechanism comprised of a heat resistant closed drying cauldron for storing night soil therein, and a heating means for heating the drying cauldron to evaporate night soil, thereby subjecting night soil to drying treatment by evaporating night soil in the airtight drying cauldron, wherein a night soil pipe for causing night soil to fluid therein is connected between the night soil tank and the drying cauldron for causing night soil inside the night soil tank to be sucked up and thrown in the drying cauldron, wherein the drying cauldron is heated to evaporate night soil in the drying cauldron, said night soil drying treatment apparatus further comprising a discharge air mechanism for sucking air and moisture evaporated in the drying cauldron, a catalyst mechanism intervened between the drying cauldron and the discharge air mechanism for subjecting elements of odor to oxidation and reduction, and an odor pipe connected between an inlet side of the catalyst mechanism and an upper space of the night soil tank for causing air to fluid therebetween, and wherein air and moisture is discharged outward by the discharge air mechanism during an evaporating drying treatment in the drying cauldron, and elements of odor are subjected to oxidation and reduction by the catalyst mechanism in the middle of discharge of air and moisture, and air remaining in the upper spaces of the night soil tank, piping and drying cauldron is sucked by the odor pipe to be passed through the catalyst mechanism, thereby rendering an odor floating in the space of the night soil tank odorless, characterized in that the catalyst cleaning mechanism is comprised of a wind pressure cleaning mechanism for supplying compressed air to the catalyst mechanism upon completion of drying treatment to blow off adherents onto the surfaces of catalysts by a wind pressure.

6. The night soil drying treatment apparatus according to claim 5, wherein the wind pressure cleaning mechanism comprises a compressor, and an air tank for storing therein compressed air supplied from the compressor and instantaneously jetting compressed air toward the catalyst mechanism when implementing cleaning treatment.

7. The night soil drying treatment apparatus according to claim 5, wherein the wind pressure cleaning mechanism is set to implement cleaning treatment every time a load of night soil is treated in the drying cauldron.

8. A catalyst mechanism of a night soil drying treatment apparatus comprising a temporary toilet provided with a night soil tank capable of storing therein night soil and a toilet stool communicating with the night soil tank at its lower portion for causing night soil to flow down, and a night soil drying treatment mechanism comprised of a heat resistant closed drying cauldron for storing night soil therein, and a heating means for heating the drying cauldron to evaporate night soil, thereby subjecting night soil to drying treatment by evaporating night soil in the airtight drying cauldron, wherein a night soil pipe for causing night soil to be fluid therein is connected between the night soil tank and the drying cauldron for causing night soil inside the night soil tank to be sucked up and thrown in the drying cauldron, wherein the drying cauldron is heated to evaporate night soil in the drying cauldron, said night soil drying treatment apparatus further comprising a discharge air mechanism for sucking air and moisture evaporated in the drying cauldron, an outer cylinder intervened between the drying cauldron and the discharge air mechanism for causing air and moisture from the drying cauldron to be fluid, a heating cylinder having a heater built therein being inserted into the outer cylinder, a space closed from an outside being formed between the outer cylinder and the heating cylinder, a catalyst being sealed in the space for subjecting elements of odor to oxidation and reduction to be odorless, whereby air and moisture including the odor sucked from the drying cauldron are heated by heat of the heater, and simultaneously caused to contact the catalyst to be subjected to oxidation and reduction to be odorless, then discharged outward.

9. The catalyst mechanism of a night soil drying treatment according to claim 8, wherein the outer cylinder and the heating cylinder are concentrically arranged, and the space closed from the outside is formed in a cylindrical shape.

10. The catalyst mechanism of a night soil drying treatment according to claim 8, wherein the catalyst is housed in a container having an outer diameter which is same as the inner diameter of the outer cylinder and an inner diameter which is same as the outer diameter of the heating cylinder, and the catalyst is inserted into the space formed by the outer cylinder and the heating cylinder together with the container.
